(19) United States
(12) Patent Application Publication
(10) Pub. No.: US 2009/0208979 A1
(43) Pub. Date: Aug. 20, 2009

(54) Method for identifying antipsychotic drug candidates

(75) Inventors: Henry SILVER, Haifa (IL); Moussa B.H. YOUDIM, Haifa (IL); Orly WEINREB, Haifa (IL)

Correspondence Address: BROWDY AND NEIMARK, P.L.L.C.;624 NINTH STREET, NW, SUITE 300, WASHINGTON, DC, 20001-5303, US

(73) Assignee: Technion Research and Development Foundation Ltd., Haifa (IL)

(21) Appl. No.: 12/372,475

(22) Filed: Feb. 17, 2009

Related U.S. Application Data
(60) Provisional application No. 61/028,716, filed on Feb. 14, 2008.

(51) Int. Cl. G01N 33/567

(52) U.S. Cl. 435/7.21

(57) ABSTRACT

The present invention provides a method for identifying a compound or a combination of compounds having a pharmacological behavior that qualifies it as a candidate for clinical development of a drug for treatment of a psychiatric disease or disorder, preferably schizophrenia. According to this method, a candidate drug is assessed for its ability to produce a biochemical profile, in either or both in vitro and in vivo test systems, which is similar to a unique reference biochemical profile obtained following treatments with drugs or drug combinations effective against both positive and negative symptoms of psychiatric diseases or disorders.

### TECHNICAL FIELD

The present invention relates to a method for identifying a compound or a combination of compounds having a pharmacological behavior that qualifies it as a candidate for clinical development of a drug for treatment of a psychiatric disease or disorder such as schizophrenia.

### BACKGROUND ART

Schizophrenia is a serious mental illness characterized by impairments in the perception or expression of reality, most commonly manifesting as auditory hallucinations, paranoid or bizarre delusions or disorganized speech and thinking in the context of significant social or occupational dysfunction. Onset of symptoms typically occurs in young adulthood, with approximately 1% of the population worldwide affected. There is a well-known tendency for schizophrenia to run in families.

Dopamine antagonist antipsychotics are the mainstay of schizophrenia treatment, but are not always effective, in particular against cognitive motivational and emotional impairments, known as “negative symptoms”, of the disease. “Atypical” antipsychotics such as clozapine, olanzapine, risperidone and ziprazidone, are arguably more effective and better tolerated than the older drugs, but their effect is also limited (Lieberman et al., 2005; Murphy et al., 2006).

The simultaneous modification of multiple neurotransmitter systems may be advantageous in complex psychiatric disorders. This approach has lead to a search for multifunctional drugs (van Hes et al., 2003) and for drug combination as a strategy to improve efficacy. A successful example of this approach for the treatment of resistant symptoms of schizophrenia, depression and obsessive-compulsive disorder (OCD) is the coadministration of selective serotonin reuptake inhibitor (SSRI) antidepressants, i.e., fluvoxamine or fluoxetine, together with antipsychotics, which produce a synergistic therapeutic effect. In schizophrenia, controlled studies showed that this combination improves negative symptoms, unresponsive to antipsychotic alone (Silver and Nassar, 1992; Spina et al., 1994; Goff et al., 1995).

Improvement in negative symptoms can be detected within two weeks of starting treatment and is not explained by any changes in depressive symptoms or extrapyramidal side effects if present (Silver and Nassar, 1992; Silver et al., 1996, 2000, 2003a; Silver and Shmugliakov, 1998). The augmenting effect is associated with the serotonergic system since maprotiline, an equally effective non-serotonergic antidepressant, did not improve negative symptoms (Silver and Shmugliakov, 1998). The mechanism of augmentation action is unknown and cannot be explained by the pharmacologic mechanisms of the individual drugs.

The development of better treatments for schizophrenia and other psychiatric diseases is limited by ignorance as to the biological causes and pathological processes. Current methods for drug screening rely on identifying candidates which mimic laboratory characteristics of drugs that are already in clinical use and, as stated above, are not effective against negative symptoms such as emotional and cognitive impairments. Such a selection results in “more of the same” types of substances and cannot lead to discovery of new drugs that are more effective against negative and cognitive symptoms than those currently available.

Likewise, more modern drug screening methods utilizing molecular markers are also forced to choose candidate substances based on promising but limited research findings and/or theoretical considerations, without an established proof of clinical effectiveness.

A further laboratory screening is limited by the fact that a given drug causes many biochemical changes, of which only some are relevant to clinical effectiveness. Since there are currently no clear criteria for differentiating biochemical changes relevant to the therapeutic response from those which are not, the clinical efficacy of a potential drug identified and developed in this way is not well predicted by the laboratory profile.

### SUMMARY OF INVENTION

In one aspect, the present invention relates to a method for identifying a compound or a combination of compounds having a pharmacological behavior that qualifies it as a candidate for clinical development of a drug for treatment of a psychiatric disease or disorder, said method comprising:
(i) treating neuronal cells expressing elements of the dopaminergic, gamma aminobutyric acid (GABA)-ergic and serotonergic systems with (a) said compound or combination of compounds; (b) a drug or drug combination effective against both positive and negative symptoms of psychiatric diseases or disorders; or (c) a control vehicle, for a sufficient time period;
(ii) measuring parameters selected from levels of proteins encoded by genes associated with expression or regulation of the GABA system, or phosphorylation levels of said proteins, in lysates or fractions thereof, obtained from said neuronal cells treated according to (i-a), (i-b) and (i-c), thus obtaining a test biochemical profile expressing the differences in said parameters between the neuronal cells treated according to (i-a) and the neuronal cells treated according to (i-c), and a reference biochemical profile expressing the differences in said parameters between the neuronal cells treated according to (i-b) and the neuronal cells treated according to (i-c); and
(iii) comparing said test biochemical profile with said reference biochemical profile,
wherein a significant similarity between said test biochemical profile and said reference biochemical profile indicates that said compound or combination of compounds has a likelihood of being a suitable candidate for clinical development of a drug for treatment of said psychiatric disease or disorder.

In another aspect, the present invention provides a kit for determining whether a compound or a combination of compounds has a pharmacological behavior that qualifies it as a candidate for clinical development of a drug for treatment of a psychiatric disease or disorder, said kit comprising:
(i) a list of parameters selected from levels of proteins encoded by genes associated with expression or regulation of the GABA system, or phosphorylation levels of said proteins;
(ii) a predetermined reference biochemical profile expressing the differences in said parameters in neuronal cells expressing elements of the dopaminergic, gamma aminobutyric acid (GABA)-ergic and serotonergic systems, treated for a sufficient time period with a drug or drug combination effective against both positive and negative symptoms of psychiatric diseases or disorders as compared with a control vehicle;
(iii) a container containing said drug or drug combination;
(iv) a set of reagents required for the detection and quantification of said parameters in neuronal cells expressing elements of the dopaminergic, gamma aminobutyric acid (GABA)-ergic and serotonergic systems, said set of reagents comprising: (a) a blotting membrane; (b) a blocking agent; (c) a primary antibody against each one of said proteins or phosphorylated form of said proteins; (d) a secondary antibody against each one of said primary antibodies, wherein said secondary antibody is linked to a detectable label; and optionally (e) a substrate for the detection of said label; and
(v) instructions for use.

In preferred embodiments, the psychiatric disease or disorder is schizophrenia.

### BRIEF DESCRIPTION OF DRAWINGS

FIG. 1 shows the relative GAD67, PKCβ, GABA_{A}β3, PKCγ and Bad mRNA expression in frontal cortices of rats chronically-treated with haloperidol, fluvoxamine, the haloperidol-fluvoxamine combination or clozapine vs. control rats. Total RNA isolated from rat frontal cortices (6 rats per group) was reverse transcribed and cDNA was amplified in real-time PCR using suitable primers. The relative expression level of each mRNA was assessed by normalizing to the reference gene 18S-rRNA. Data (mean±SEM) is expressed as percent of control set as 100%. Student's t-test * p<0.05; ** p<0.01 drug group compared with control group.

FIGS. 2A-2C show the relative GABA_{A}β3 protein expression in the whole tissue lysate (2A), cytosolic (2B) and membranal compartments (2C) of frontal cortices of rats chronically-treated with haloperidol (Halo), fluvoxamine (Flu), the haloperidol-fluvoxamine combination (H+F) or clozapine (Cloz) vs. control rats (Cont). Protein samples from individual frontal cortices were subjected to subcellular fractionation and consequent Western blot analysis using primary antibodies against GABA_{A}β3. Immunoreactive bands were analyzed by densitometry and normalized against β-actin levels. Percent of control values are given as mean±SEM from 6-12 rats, controls are expressed as 1. t-test * p<0.05; ** p<0.01 drug group vs. control group.

FIGS. 3A-3D show the relative GAD67 (3A), PKCβ2 (3B), ERK1 (3C) and ERK2 (3D) protein expression in whole tissue lysates of frontal cortices of rats chronically-treated with haloperidol (Halo), fluvoxamine (Flu), a combination thereof (H+F) or clozapine (Cloz) vs. control rats (Cont). Whole tissue lysates from individual frontal cortices were subjected to Western blot analysis using primary antibodies. Immunoreactive bands were analyzed by densitometry and normalized against β-actin levels. Percent of control values are given as mean±SEM from 6-12 rats, controls are expressed as 1. t-test * p<0.05; ** p<0.01; *** p<0.001 drug group vs. control group.

FIGS. 4A-4C show the relative PKC phosphorylation in whole tissue lysates (4A), membranal (4B) and cytosolic compartments (4C) of frontal cortices of rats chronically-treated with haloperidol (Halo), fluvoxamine (Flu), a combination thereof (H+F) or clozapine (Cloz) vs. control rats (Cont). Protein samples from individual frontal cortices were subjected to subcellular fractionation and consequent Western blot analysis of the whole lysate, cytosolic or membranal compartments, using primary antibodies against phospho-PKC-pan.

Immunoreactive bands were analyzed by densitometry and normalized against β-actin levels. Percent of control values are given as mean±SEM from 6-12 rats. t-test * p<0.05 drug group vs. control group.

FIGS. 5A-5B show the relative phosphorylation level of ERK1 (5A) and ERK2 (5B) in whole tissue lysates of frontal cortices of rats chronically-treated with haloperidol (Halo), fluvoxamine (Flu), a combination thereof (H+F) or clozapine (Cloz) vs. control rats (Cont). Whole tissue lysates from individual frontal cortices were subjected to Western blot analysis using primary antibodies. Immunoreactive bands were analyzed by densitometry and normalized against ERK1 and ERK2 levels. Percent of control values are given as mean±SEM from 6-12 rats. t-test * p<0.05; ** p<0.01 drug group vs. control group.

FIGS. 6A-6D show the relative GABA_{A}β3 protein expression in cytosolic (6A, 6C) and membranal compartments (6B, 6D) of frontal cortices of rats administered with a single intraperitoneal (IP) injection of haloperidol (Halo), fluvoxamine (Flu), the haloperidol-fluvoxamine combination (H+F) or clozapine (Cloz) vs. control rats (Cont), and sacrificed 30 minutes (6A, 6B) or 1 hr (6C,6D) later. Protein samples from individual frontal cortices were subjected to subcellular fractionation and consequent Western blot analysis using primary antibodies against GABA_{A}β3. Immunoreactive bands were analyzed by densitometry and normalized against β-actin levels. Percent of control values are given as mean±SEM from 8-12 rats, controls are expressed as 1. t-test * p<0.05; ** p<0.01 drug group vs. control group.

FIGS. 7A-7D show the relative PKCβ2 protein expression in cytosolic (7A, 7C) and membranal compartments (7B, 7D) of frontal cortices of rats administered with a single IP injection of haloperidol (Halo), fluvoxamine (Flu), the haloperidol-fluvoxamine combination (H+F) or clozapine (Cloz) vs. control rats (Cont), and sacrificed 30 minutes (7A, 7B) or 1 hr (7C,7D) later. Protein samples from individual frontal cortices were subjected to subcellular fractionation and consequent Western blot analysis using primary antibodies against PKCβ2. Immunoreactive bands were analyzed by densitometry and normalized against β-actin levels. Percent of control values are given as mean±SEM from 8-12 rats, controls are expressed as 1. t-test * p<0.05; ** p<0.01 drug group vs. control group.

FIGS. 8A-8D show relative phosphorylation level of ERK2 in cytosolic (8A, 8C) and membranal compartments (8B, 8D) of frontal cortices of rats administered with a single IP injection of haloperidol (Halo), fluvoxamine (Flu), the haloperidol-fluvoxamine combination (H+F) or clozapine (Cloz) vs. control rats (Cont), and sacrificed 30 minutes (8A, 8B) or 1 hr (8C,8D) later. Protein samples from individual frontal cortices were subjected to subcellular fractionation and consequent Western blot analysis using primary antibodies against phospho-ERK2. Immunoreactive bands were analyzed by densitometry and normalized against total ERK2 levels. Percent of control values are given as mean±SEM from 8-12 rats, controls are expressed as 1. t-test * p<0.05; ** p<0.01; *** p<0.001 drug group vs. control group.

FIGS. 9A-9B show the relative phosphorylation level of GABA_{A}β2/β3 subunit in lysates obtained from primary cortical neuronal cell cultures treated for 15 minutes (9A) or 7 days (9B) with haloperidol (Halo), fluvoxamine (Flu), a combination thereof (H+F) or clozapine (Cloz) vs. vehicle (Cont). Whole tissue lysates from individual frontal cortices were subjected to Western blot analysis using primary antibodies. Immunopresipitation with anti-GABA_{A}β2/β3 antibody was performed, and the obtained lysates were subjected to Western blot analysis using primary antibodies against phospho-serine. Immunoreactive bands were analyzed by densitometry and normalized against total GABA_{A}β2/β3 levels. Percent of control values are given as mean±SEM, controls are expressed as 1. The data is representative results of 2-3 experiments. t-test * p<0.05; ** p<0.01 drug treatment vs. control.

FIG. 10 shows the relative phosphorylation level of GABA_{A}β2/β3 subunit in lysates obtained from primary cortical neuronal cell cultures pretreated for 30 minutes with the non-selective PKC inhibitor GF109203X, and then treated for 15 minutes with haloperidol (Halo), fluvoxamine (Flu), a combination thereof (H+F) or clozapine (Cloz) vs. vehicle (Cont). Immunoprecipitation with anti-GABA_{A}β2/β3 antibodies was performed, and the obtained lysates were subjected to Western blot analysis using primary antibodies against phospho-serine. Immunoreactive bands were analyzed by densitometry and normalized against total GABA_{A}β2/β3 levels. Percent of control values are given as mean±SEM. The data is representative results of 2-3 experiments.

FIGS. 11A-11C show the differential regulation of PKC protein level and activation by haloperidol (Halo), fluvoxamine (Flu), a combination thereof (H+F) or clozapine (Cloz) vs. vehicle (Cont) in cultured cortical neurons. Primary cortical neuron cultures were treated for 15 minutes (11A,11B) or 7 days (11C) with the various drugs. Whole cell lysates were subjected to Western blot analysis using primary antibodies against phospho-PKC-pan (11A) or PKCβ2 (11B,11C). Immunoreactive bands were analyzed by densitometry and normalized against β-actin levels. Percent of control values are given as mean±SEM, controls are expressed as 1. The data is representative results of 2-3 experiments. t-test * p<0.05; ** p<0.01 drug treatment vs. control.

FIG. 12 shows the relative phosphorylation level of GABA_{A}β2/β3 subunit in lysates obtained from primary cortical neuronal cell cultures pretreated for 30 minutes with the non-selective ERK inhibitor PD98059, and further treated for 15 minutes with haloperidol (Halo), fluvoxamine (Flu), a combination thereof (H+F) or clozapine (Cloz) vs. vehicle (Cont). Immunoprecipitation with anti-GABA_{A}β2/β3 antibodies was performed, and the obtained lysates were subjected to Western blot analysis using primary antibodies against phospho-serine. Immunoreactive bands were analyzed by densitometry and normalized against total GABA_{A}β2/β3 levels. Percent of control values are given as mean±SEM. The data is representative results of 2-3 experiments. t-test ** p<0.01 drug treatment vs. control.

FIGS. 13A-13D show the relative phosphorilation level (13A,13B) and the protein level (13C,13D) of ERK in whole cell lysates obtained from primary cortical neuronal cell cultures pretreated for 15 minutes (acute treatment, 13A, 13C) or 7 days (chronic treatment, 13B, 13D) with haloperidol (Halo), fluvoxamine (Flu), a combination thereof (H+F) or clozapine (Cloz) vs. vehicle (Cont). Cell lysates were subjected to Western blot analysis using primary antibodies against phospho-ERK1/2 (13A,13B) or ERK1/2 (13C,13D). Immunoreactive bands were analyzed by densitometry and normalized against total ERK2 (13A,13B) or β-actin (13C,13D) levels. Percent of control values are given as mean±SEM, controls are expressed as 1. The data is representative results of 2-3 experiments. t-test * p<0.05; ** p<0.01 drug treatment vs. control.

FIGS. 14A-14B show the differential regulation of GAD67 protein level by haloperidol (Halo), fluvoxamine (Flu), a combination thereof (H+F) or clozapine (Cloz) vs. vehicle (Cont) in primary cortical neuronal cell cultures. Primary cortical neuronal cell cultures were treated for 15 minutes (acute treatment, 14A) or 7 days (chronic treatment, 14B) with the various drugs. Whole cell lysates were subjected to Western blot analysis using primary antibodies against GAD67. Immunoreactive bands were analyzed by densitometry and normalized against β-actin levels. Percent of control values are given as mean±SEM. The data is representative results of 2-3 experiments. t-test * p<0.05; ** p<0.01 drug treatment vs. control.

### MODES FOR CARRYING OUT THE INVENTION

In one aspect, the present invention relates to a method for identifying a compound or a combination of compounds having a pharmacological behavior that qualifies it as a candidate for clinical development of a drug for treatment of a psychiatric disease or disorder, as defined above.

The actions of neurotransmitters at synapses throughout the brain arise from the tremendous diversity of postsynaptic neurotransmitter receptors, which are proteins embedded in the plasma membranes of postsynaptic cells. These receptors translate chemical signals into electrical signals by binding neurotransmitter molecules secreted by presynaptic neurons, which lead in turn to opening or closing of postsynaptic ion channels. The postsynaptic currents produced by the synchronous opening or closing of ion channels changes the conductance of the postsynaptic cell, thus increasing or decreasing its excitability.

The dopaminergic, GABA-ergic and serotonergic systems comprise elements, or components, involved in the synthesis and release of dopamine, GABA and serotonin, respectively, from presynaptic neurons as well as dopamine, GABA and serotonin receptors and other elements, involved in signal transduction in postsynaptic cells.

It is well established that GABA_{A} receptor activity and synaptic stability can be modulated by phosphorylation and receptor trafficking. Several kinase molecules, e.g., protein kinase C (PKC), c-AMP dependent protein kinase also known as protein kinase A (PKA), Ca²⁺/calmodulin dependent protein kinase II (CamKII), and extracellular signal-regulated kinase (ERK) are implicated in GABA_{A} subunit phosphorylation and demonstrated to modulate receptor activity. Fast modulation of GABA_{A} receptor activity by phosphorylation might be important to the reciprocal regulative interactions between 5-hydroxytryptamine (5-HT), dopamine (DA) and GABA in neurons, while PKC and PKA play a central role in this cross-talk between neurotransmitter systems.

The term “neuronal cells expressing elements of the dopaminergic, GABA-ergic and serotonergic systems”, as used herein, refers to any neuronal cells expressing certain elements related to the dopaminergic, GABA-ergic and serotonergic systems as listed hereinabove. Particular examples of such neuronal cells include, without being limited to, neuronal cell or primary neuronal cell cultures obtained from various parts of the brain.

In one embodiment, the method of the present invention is performed in vitro, and the neuronal cells expressing elements of the dopaminergic, GABA-ergic and serotonergic systems are cortical neuronal cell cultures, preferably primary cortical neuronal cell cultures.

In another embodiment, the first step of the method of the present invention is performed in vivo, and the neuronal cells expressing elements of the dopaminergic, GABA-ergic and serotonergic systems are neuronal cells obtained from a cortex, preferably from a frontal cortex, more preferably from a prefrontal cortex of a mammal such as a rodent, e.g., a rat or a mouse, administered with said compound or combination of compounds, said drug or drug combination, or said control vehicle.

The term “genes associated with expression or regulation of the GABA system” refers to any gene associated with the GABA-ergic system as defined above. Examples of such genes, without being limited to, include GABA_{A} β3 receptor (GABA_{A} Rβ3 or GABA_{A}β3), glutamic acid decarboxylase 67 (GAD67), a protein kinase C (PKC) isoform, preferably PKCβ and PKCγ, extracellular signal-regulated kinase 1 (ERK1), extracellular signal-regulated kinase 2 (ERK2), receptor of activated protein kinase C 1 (Rack1), serine/threonine kinase glycogen synthase kinase-3 (GSK-3), a protein kinase A (PKA) isoform, 5-hydroxytriptamine receptor (5-HTR), dopamine receptor (DAR), metabotropic glutamate receptor (mGLUR), N-methyl-D-aspartate receptor (NMDAR), adenylate cyclase (AC), diacylglycerol (DAG) receptor, and phospholipase C (PLC) receptor.

Fast modulation of GABA_{A} receptor activity by phosphorylation might be important to the reciprocal regulative interactions between 5-HT, DA and GABA in neurons, while PKC and PKA play a central role in this cross-talk between neurotransmitter systems, as shown in Scheme 1 hereinafter. Previous studies describe 5-HT₂ receptor activation-induced GABA_{A} phosphorylation by PKC (Feng et al., 2001), while 5-HT₄ agonists exhibit activity-dependent bidirectional regulation of GABA_{A} activity by PKA (Cai et al., 2002). On the presynaptic side, GABAergic inhibition is regulated by 5-HT through the activation of 5-HT₂, 5-HT₁ and 5-HT₃ receptors on GABAergic intereneurons (Yan, 2002). D₃ and D₄ selective agonists have been shown to reduce postsynaptic GABA_{A} receptor currents via PKA activation (Wang et al., 2002; Chen et al., 2006).

The compound or combination of compounds being evaluated, according to the method of the present invention, for treatment of a psychiatric disease or disorder may be either a drug or drug combination approved for treatment of humans against an indication other than psychiatric disease or disorder, or a chemical molecule or combination of molecules currently being evaluated as a potential drug for treatment of a psychiatric disease or disorder.

The term “drug or drug combination effective against both positive and negative symptoms of psychiatric diseases or disorders” or “reference drug or drug combination”, used herein interchangeably, refers to any drug or drug combination that is effective against both positive symptoms, i.e., hallucinations, delusions and racing thoughts, which generally respond to antipsychotic medicines, as well as negative symptoms, i.e., apathy, lack of emotion and poor or nonexistant social functioning, associated with the psychiatric disease or disorder. In view of these properties, such drug or drug combination can thus principally be used in treating patients with treatment-resistant schizophrenia, a term generally used for the failure of symptoms to satisfactorily respond to at least two different antipsychotics.

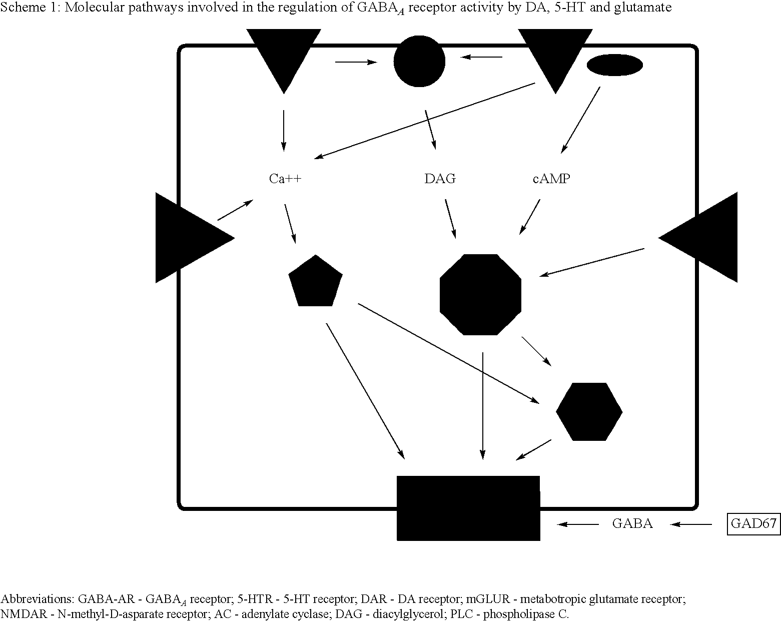

In one embodiment, the drug combination effective against both positive and negative symptoms of psychiatric diseases or disorders is a combination of an antipsychotic agent and an antidepressant agent functioning pharmacologically as a selective serotonin reuptake inhibitor (SSRI).

Non-limiting examples of antipsychotic agents include the atypical antipsychotic drugs risperidone (Risperdal®), olanzapine (Zyprexa®), ziprasidone (Geodone®) and clozapine; the typical antipsychotic drugs haloperidol, perphenazine and trifluperazine (Eskazinyl®); the antipsychotic drug amisulpride (Solian®); and a thioxanthene derivative such as the typical antipsychotic drugs chlorprothixene and thiothixene (Navane®), and the typical antipsychotic neuroleptic drugs flupentixol (Depixol® or Fluanxol®) and zuclopenthixol (Cisordinol®, Clopixol® or Acuphase®), available as zuclopenthixol decanoate, zuclopenthixol acetate and zuclopenthixol dihydrochloride.

Examples of antidepressant agents, without limitation, include fluoxetine, an antidepressant of the SSRI class (Prozac®); or fluvoxamine, an antidepressant which functions pharmacologically as an SSRI (Luvox®).

In preferred embodiments, the drug or drug combination effective against both positive and negative symptoms of psychiatric diseases or disorders is a combination of the typical antipsychotic drug haloperidol and the antidepressant agent fluvoxamine; or the atypical antipsychotic drug clozapine, which is effective against both positive and negative symptoms of schizophrenia. In a more preferred embodiment, the reference drug or drug combination is a combination of haloperidol and fluvoxamine.

The protein levels of the genes associated with expression or regulation of the GABA system, or the phosphorylation levels of said proteins, are measured in lysates, or fractions thereof, obtained from the neuronal cells as defined above, using any suitable technique known in the art, e.g., as described in detail in Materials and Methods hereinafter. The protein levels or protein phosphorylation levels may be measured in neuronal cell whole lysates which may be obtained, e.g., by homogenizing the neuronal cells in a suitable buffer, centrifugation of the homogenate at low speed and recovery of the supernatant as described in Materials and Methods hereinafter. When it is necessary to evaluate whether receptor endocytosis is induced following the treatment with the compound or combination of compounds evaluated, the protein levels or protein phosphorylation levels may further be measured in the cytosolic and membranal compartments, or fractions, which may be separated from the whole lysate as described in Materials and Methods hereinafter. The terms “total protein level” and “total phosphorylation level” with respect to a certain gene refer to the protein level of said gene or to the phosphorylation level of said protein, respectively, in the unfractionated lysate.

The control vehicle used according to the method of the present invention may be any suitable control vehicle but it is preferably the solution in which the compound or combination of compounds being evaluated, or the reference drug or drug combination, are dissolved, as described in Materials and Methods.

The term “sufficient time period”, as used herein, refers to a period of time from minutes to several days or more, during which the neuronal cells are treated, according to step (i) of the method of the present invention, with either the compound or combination of compounds being evaluated, or the reference drug or drug combination. The treatment of the neuronal cells may be either acute or chronic. In certain embodiments, step (i) of the method of the present invention is performed in vivo; the mammal from which the neuronal cells are obtained is administered with a single injection of either the compound or combination of compounds being evaluated, or the reference drug or drug combination; and the neuronal cells are obtained, e.g., about 30 or 60 minutes following administration, to identify the early-course of the certain treatment-induced changes in the various parameters of interest. In other embodiments, step (i) of the method of the present invention is performed in vivo; the mammal from which the neuronal cells are obtained is chronically treated with either the compound or combination of compounds being evaluated, or the reference drug or drug combination, and the neuronal cells are obtained, e.g., following treatment of about 14 days, a period often used in chronic administration studies in animals, or more. In still other embodiments, the method of the present invention is performed in vitro, and the neuronal cells are treated with either the compound or combination of compounds being evaluated, or the reference drug or drug combination, for a period of time simulating either acute or chronic treatment, e.g., during about 15, 30, 45 or 60 minutes, or about 7 days or more, respectively.

As described hereinafter, the changes in the protein levels of certain genes associated with expression or regulation of the GABA system, or in the phosphorylation levels of said proteins, following a chronic administration of a certain compound or combination of compounds are not always consistent with the corresponding changes following an acute administration of the same compound or combination of compounds, probably due to, inter alia, the time dependent-effects, -regulation and -dynamic alterations in cell signaling pathways. Thus, it should be understood that in certain embodiments, the measuring of each one of the parameters according to step (ii) of the method of the present invention is performed following treatment of the neuronal cells for a different time period. For example, while some of the parameters may be measured following an acute treatment of the cells, other parameters may be measured following a chronic treatment of the cells. Obviously, the measurement of each one of the parameters is performed after a time period that is identical for cells treated with the compound or combination of compounds; the drug or drug combination; or the control vehicle.

The term “biochemical profile”, as used herein, refers to a profile showing the combination of specific differences observed in the protein levels and/or protein phosphorylation levels of a certain group of genes associated with expression or regulation of the GABA system, measured in lysates or fractions thereof obtained from the neuronal cells defined above, following treatment for a sufficient time period with either the compound or combination of compounds being evaluated according to the method of the present invention; or with a drug or drug combination effective against both positive and negative symptoms of psychiatric diseases or disorders, compared to control neuronal cells, treated with a control vehicle. In particular, the biochemical profile obtained following treatment with the compound or combination of compounds being evaluated is referred herein as “a test biochemical profile”; and the biochemical profile obtained following treatment with a reference drug or drug combination is referred herein as “a reference biochemical profile”.

The biochemical profile as defined herein may comprise various and different combinations of specific changes observed in the protein levels of selected genes associated with expression or regulation of the GABA system, and/or in the phosphorylation level of said proteins, measured as described above. Furthermore, in certain cases, the reference biochemical profile may be restricted to a combination of specific changes mutually observed following a separate treatment with more than one reference drug or drug combination, e.g., following a treatment with a combination of haloperidol and fluvoxamine as well as with clozapine.

The group of genes associated with expression or regulation of the GABA system may include any combination of at least two, preferably at least three, more preferably at least four, such genes, hence the biochemical profile as defined above is based on at least two different parameters selected from protein level of each one of said genes and/or phosphorylation levels of said proteins. For illustration, a theoretical biochemical profile may be based on two genes designated A and B, both associated with expression or regulation of the GABA system, and may be defined as certain combinations of parameters, e.g., an increase in the protein levels of A and B and a decrease in the protein phosphorylation level of A; a decrease in the protein level of A and an increase in the protein phosphorylation levels of both A and B; a decrease in the protein level of A, a decrease in the membranal fraction A protein level, an increase in the cytosolic fraction A protein level and an increase in the protein phosphorylation level of B; etc.

In all cases, protein levels are measured and compared with the level of a control protein which is not influenced neither by the compound or combination of compounds evaluated nor by the reference drug or drug combination. Non-limiting examples of such control proteins include β-actin, β-tubulin and glyceraldehyde 3-phosphate dehydrogenase (GAPDH). In a preferred embodiment, the control protein is β-actin.

A significant difference with respect to a certain parameter is determined in a case wherein there is a statistically significant difference between the level of that parameter in the neuronal cells treated with the compound or combination of compounds evaluated vs. the control neuronal cells, or in the neuronal cells treated with the reference drug or drug combination vs. the control neuronal cells. The statistical analysis may be performed using any suitable statistic test such as Student's t-test with an a value of, e.g., 5%, 1% or 0.1%.

The term “significant similarity between the biochemical profiles” refers to a situation in which the pattern of differences observed in the test biochemical profile with respect to at least 50%, preferably at least 60%, more preferably at least 75%, most preferably at least 90%, of the parameters measured is identical to the pattern of differences observed with respect to said parameters in the reference biochemical profile. In fact, the likelihood of the compound or combination of compounds evaluated being a suitable candidate for clinical development of a drug for treatment of a psychiatric disease or disorder is considered to increase with the increase in the number of parameters which are altered in the direction defined by the reference biochemical profile, wherein a total similarity between the profiles indicates a very high likelihood. For illustration, a theoretical test biochemical profile, which is based on two genes designated A and B, and is characterized by an increase in the protein levels of A and B, and a decrease in the protein phosphorylation level of A, will be of a significant similarity, in particular a total similarity, to a reference biochemical profile, provided that the reference biochemical profile is characterized by an increase in the protein levels of A and B, and a decrease in the protein phosphorylation level of A as well.

As described hereinabove, each one of the parameters being included in the biochemical profile according to the method of the present invention is measured in lysates, or fractions thereof, obtained from the neuronal cells defined above following treatment with the compound or combination of compounds being evaluated; or with a reference drug or drug combination, and is then compared to its corresponding parameter measured in lysates, or fractions thereof, obtained from control neuronal cells, treated with a control vehicle for the same period of time.

As shown in the Examples section hereinafter, treatment with a selective serotonin reuptake inhibitor (SSRI)-antipsychotic combination results in biochemical changes which are substantially different from those observed following administration of each one of the individual drugs separately, and these unique changes may be directly related to the therapeutic mechanism of action of that drug combination. As further shown, the atypical antipsychotic drug clozapine, which also ameliorates negative symptoms in schizophrenia patients but has a pharmacological action that is different from both haloperidol and fluvoxamine, produces a biochemical profile that is similar to that obtained following the treatment with the haloperidol-fluvoxamine combination.

As particularly shown in Example 2, chronic treatment of rats with both the haloperidol-fluvoxamine combination and clozapine resulted in a significant decrease in the GABA_{A} Rβ3 protein level in the frontal cortical neuronal cells. The change in the GABA_{A} Rβ3 protein level was accompanied by receptor endocytosis, as deduced from an increase in the cytosolic fraction GABA_{A} Rβ3 protein level and a decrease in the membranal fraction GABA_{A} Rβ3 protein level. As further shown in Example 7, both acute and chronic in vitro treatments of primary cortical neuronal cells with the haloperidol-fluvoxamine combination and clozapine significantly increased the GABA_{A} Rβ3 protein phosphorylation level, whereas pretreatment of the neuronal cells with a non-selective PKC inhibitor prevented this effect, as shown in Example 8. As further shown in Example 10, the increase of GABA_{A} Rβ3 protein phosphorylation level remained unaltered following pretreatment of the neuronal cells with a non-selective inhibitor of the mitogen-activated protein kinase (MAPK) pathway, ERK1/2, indicating that ERK is not involved in the modulation of GABA_{A} Rβ3 phosphorylation by said drugs; however, may be affected by GABA_{A} Rβ3 phosphorylation.

Example 3 shows that chronic treatment of rats with both the haloperidol-fluvoxamine combination and clozapine resulted in a significant decrease in the PKCβ2 protein level in the frontal cortical neuronal cells. This finding is further supported by Example 9, showing that PKCβ2 protein level was significantly decreased by both acute and chronic in vitro treatments of primary cortical neuronal cell cultures with clozapine. As further shown in Examples 3 and 5, chronic treatment of rats with both the haloperidol-fluvoxamine combination and clozapine resulted in a significant increase in both the ERK1 and ERK2 protein levels, accompanied by a significant decrease in the phosphorylation level of said proteins, in the frontal cortical neuronal cells.

Interestingly, the changes in the protein levels of certain genes associated with expression or regulation of the GABA system, or in the phosphorylation levels of said proteins, following a chronic administration of both the haloperidol-fluvoxamine combination and clozapine, were not always consistent with the changes in the protein levels of said genes or in the phosphorylation levels of said proteins following an acute administration of the aforesaid drug or drug combination. These differences may reflect the time dependent-effects, -regulation and -dynamic alterations in cell signaling pathways, as well as the diverse interactions among molecular cascades in response to the drug treatment the chronic vs. the acute paradigms.

As described hereinabove, differences unique to the haloperidol-fluvoxamine combination were identified both in neuronal cells obtained from frontal cortices of rats treated with the aforesaid drug combination, as well as in primary neuronal cell cultures treated with said drug combination in vitro. These differences involved a range of substances relevant to neuronal cell signaling, growth and integrity, and included proteins and neurotransmitter metabolism parameters.

Thus, in one preferred embodiment of the present invention, the genes associated with expression or regulation of the GABA system are GABA_{A} Rβ3, PKCβ2, ERK1 and ERK2; and the various parameters measured are total GABA_{A} Rβ3 protein level, cytosolic fraction GABA_{A} Rβ3 protein level, membranal fraction GABA_{A} Rβ3 protein level, total GABA_{A} Rβ3 phosphorylation level, total PKCβ2 protein level, total ERK1 protein level, total ERK1 phosphorylation level, total ERK2 protein level and total ERK2 phosphorylation level.

In a most preferred embodiment, the reference biochemical profile to which the test biochemical profile is compared comprises a decrease in the total GABA_{A} Rβ3 protein level; an increase in the total GABA_{A} Rβ3 phosphorylation level; a decrease in the membranal fraction GABA_{A} Rβ3 protein level; an increase in the cytosolic fraction GABA_{A} Rβ3 protein level; a decrease in the total PKCβ2 protein level; an increase in both the total ERK1 and the total ERK2 protein levels; and a decrease in both the total ERK1 and the total ERK2 phosphorylation levels, and the neuronal cells are cortical neuronal cell culture treated with said drug or drug combination for a time period of about 7 days or more, or neuronal cells obtained from a cortex, preferably a frontal cortex, more preferably a prefrontal cortex, of a mammal administered with said drug or drug combination for a time period of about 14 days or more.

The advantage of the method of the present invention is that it is based on proven clinical effectiveness against both positive symptoms as well as negative symptoms not responsive to standard antipsychotic treatment. The selection criteria are based on the principle that the biochemical effects common to pharmacologically distinct but equally effective clinical treatments are directly related to the biochemical mechanisms resulting in clinical improvement. The method of the present invention therefore enables differentiation in the laboratory of clinically relevant effects of drugs from those not relevant. Compounds or combinations of compounds producing, following administration to neuronal cells as defined above, a biochemical profile similar to a reference profile produced by a clinically approved drug or drug combination are likely drug candidates to be effective against both positive and negative symptoms.

In other words, the method of the present invention uses proven clinical effectiveness against both positive symptoms as well as negative symptoms of schizophrenia, resistant to currently available standard treatments, as the ultimate criterion for identifying therapeutically relevant biochemical changes. The concept of the invention is based on the principle that biochemical changes common to pharmacologically distinct but clinically equally effective treatments against both positive and negative symptoms are directly related to the molecular mechanisms responsible for that therapeutic effectiveness. In particular, it is proposed that the pattern of biochemical changes, which characterizes the combined SSRI-antipsychotic treatment and is different from the effects of each individual drug, identifies the biochemical changes involved in the mechanisms of the therapeutic effect against both positive and negative symptoms of a psychiatric disease or disorder such as schizophrenia. This pattern is therefore used as a reference biochemical profile for identification of new compounds or combinations of compounds having a potential for therapeutic effectiveness against a psychiatric disease or disorder.

Practically, the method of the present invention consists of a set of in vitro and/or in vivo tests, used to identify potential new drugs, wherein a candidate drug is assessed for its ability to produce a biochemical profile, in either or both in vitro and in vivo test systems, which is similar to a unique reference biochemical profile obtained following treatments with drugs or drug combinations effective against both positive and negative symptoms of psychiatric diseases or disorders, e.g., the haloperidol-fluvoxamine combination, the atypical antipsychotic drug clozapine, or both.

The psychiatric disease or disorder according to the present invention may be any psychiatric or neuropsychiatric disease or disorder which includes disturbances in motivational, emotional or cognitive function, i.e., “negative symptoms”, as part of the clinical syndrome, such as schizophrenia, obsessive-compulsive disorder (OCD), major depression, bipolar disorder or dementia accompanied, i.e., complicated, by aggression or affective disorder, namely mental disorder characterized by dramatic changes or extremes of mood, such as manic (elevated, expansive or irritable mood with hyperactivity, pressured speech and inflated self-esteem), depressive (dejected mood with disinterest in life, apathy, sleep disturbance, agitation and feelings of worthlessness or guilt) episodes, or combinations thereof. In a preferred embodiment, the psychiatric disease or disorder is schizophrenia.

In view of the aforesaid, the present invention particularly relates to a method for identifying a compound or a combination of compounds having a pharmacological behavior that qualifies it as a candidate for clinical development of a drug for treatment of schizophrenia, said method comprising:
(i) treating neuronal cells expressing elements of the dopaminergic, GABAergic and serotonergic systems with (a) said compound or combination of compounds; or (b) a control vehicle, wherein said neuronal cells are cortical neuronal cell culture treated for a time period of about 7 days or more, or neuronal cells obtained from a cortex, preferably a frontal cortex, more preferably a prefrontal cortex, of a mammal administered with (a) or (b), for a time period of about 14 days or more;
(ii) measuring total levels of proteins encoded by the genes GABA_{A} Rβ3, PKCβ2, ERK1 and ERK2, cytosolic fraction GABA_{A} Rβ3 protein level, membranal fraction GABA_{A} Rβ3 protein level, and phosphorylation levels of GABA_{A} Rβ3, ERK1 and ERK2 in lysates, or fractions thereof, obtained from said neuronal cells treated according to (i-a) and (i-b); and
(iii) comparing the levels obtained in (ii) for the neuronal cells treated according to (i-a) and for the neuronal cells treated according to (i-b), wherein a decrease in the total GABA_{A} Rβ3 protein level; an increase in the total GABA_{A} Rβ3 phosphorylation level; a decrease in the membranal fraction GABA_{A} Rβ3 protein level; an increase in the cytosolic fraction GABA_{A} Rβ3 protein level; a decrease in the total PKCβ2 protein level; an increase in both the total ERK1 and the total ERK2 protein levels; and a decrease in both the total ERK1 and the total ERK2 phosphorylation levels in the neuronal cells treated according to (i-a) in comparison to that of the neuronal cells treated according to (i-b) indicate that said compound or combination of compounds has a likelihood of being a suitable candidate for clinical development of a drug for treatment of schizophrenia.

In another aspect, the present invention provides a kit for determining whether a compound or a combination of compounds has a pharmacological behavior that qualifies it as a candidate for clinical development of a drug for treatment of a psychiatric disease or disorder, said kit comprising:
(i) a list of parameters selected from levels of proteins encoded by genes associated with expression or regulation of the GABA system, or phosphorylation levels of said proteins;
(ii) a predetermined reference biochemical profile expressing the differences in said parameters in neuronal cells expressing elements of the dopaminergic, gamma aminobutyric acid (GABA)-ergic and serotonergic systems, treated for a sufficient time period with a drug or drug combination effective against both positive and negative symptoms of psychiatric diseases or disorders as compared with a control vehicle;
(iii) a container containing said drug or drug combination;
(iv) a set of reagents required for the detection and quantification of said parameters in neuronal cells expressing elements of the dopaminergic, gamma aminobutyric acid (GABA)-ergic and serotonergic systems, said set of reagents comprising: (a) a blotting membrane; (b) a blocking agent; (c) a primary antibody against each one of said proteins or phosphorylated form of said proteins; (d) a secondary antibody against each one of said primary antibodies, wherein said secondary antibody is linked to a detectable label; and optionally (e) a substrate for the detection of said label; and
(v) instructions for use.

The kit of the present invention can be used for carrying out the method defined above, utilizing a certain compound or combination of compounds to be evaluated, and neuronal cells expressing elements of the dopaminergic, GABA-ergic and serotonergic systems, such as neuronal cell or primary neuronal cell cultures obtained from various parts of the brain, as defined above. In particular, cultures of said neuronal cells may be treated in vitro with the compound or combination of compounds being evaluated, or alternatively, a mammal such as a rodent, e.g., a rat or a mouse, can be administered with said compound or combination of compounds, and neuronal cells as defined above may then be obtained from the cortex, preferably from the frontal cortex, more preferably from the prefrontal cortex, of said mammal.

As described above, the method of the present invention is based on the creation of two biochemical profiles, i.e., a test biochemical profile and a reference biochemical profile, which are then compared for significant similarity. In particular, these biochemical profiles show the combination of specific differences observed in the protein levels and/or protein phosphorylation levels of a certain group of genes associated with expression or regulation of the GABA system, measured in lysates or fractions thereof obtained from the neuronal cells defined above, following treatment for a sufficient time period with the compound or combination of compounds being evaluated; and with a reference drug or drug combination, respectively, compared to control neuronal cells, treated with a control vehicle.

A container, e.g., an ampoule or vial, containing the drug or drug combination effective against both positive and negative symptoms of psychiatric diseases or disorders, i.e., the reference drug or drug combination required for the preparation of the reference biochemical profile, is provided as a part of the kit of the present invention. As defined above, the kit further includes a list of parameters selected from protein or phosphoprotein levels of genes associated with expression or regulation of the GABA system, as well as a predetermined reference biochemical profile, consisting of the parameters listed, for comparison with the reference biochemical profile obtained following treatment of the neuronal cells with the reference drug or drug combination provided as compared to control neuronal cells treated with a control vehicle.

In order to produce the test biochemical profile and the reference biochemical profile, the neuronal cells are treated as defined in step (i) of the method of the present invention, and the specific parameters listed, i.e., the protein or phosphoprotein levels of certain genes associated with expression or regulation of the GABA system, are measured using the various reagents provided as parts of the kit. The various parameters are measured in lysates, or fractions thereof, obtained from the neuronal cells as defined above, using any suitable technique known in the art, e.g., dot-blot or Western blot as described in Materials and Methods hereinafter.

The set of reagents provided as a part of the kit of the present invention comprises (i) a blotting membrane for binding or immobilizing the proteins present in the lysates, or fractions thereof, obtained from the neuronal cells; (ii) a blocking agent for reducing non-specific binding of the antibodies used when carrying out the method of the present invention; primary antibodies against the various proteins or phosphoproteins measured as per the list of parameters that should be detected and quantified; secondary antibodies against the various primary antibodies provided, wherein each one of said secondary antibody is linked to a detectable label; and optionally a substrate for the detection of that label.

The blotting membrane may be any membrane commonly used in Western- and dot-blotting procedures, such as a nitrocellulose membrane.

The blocking agent may be any blocking agent commonly used in Western- and dot-blotting procedures, such as milk powder or bovine serum albumin (BSA).

The primary antibodies may be any monoclonal or polyclonal antibodies suitable for use when carrying out the method of the present invention. These antibodies may be either commercially available, e.g., the primary antibodies used in the examples described herein, or specifically prepared for use in that kit.

The secondary antibodies may be any polyclonal antibody suitable for use when carrying out the method of the present invention, e.g., commercially available polyclonal antibodies such as those used in the examples described herein. As stated above, these antibodies are linked to a detectable label. Non-limiting examples of such labels include fluorophores, metal nanoparticles and enzymes such as horseradish peroxide (HRP) and alkaline phosphatase.

In case the label linked to the secondary antibodies is an enzyme, a substrate for the detection of the enzyme is included in the set of reagents provided with the kit of the present invention. This substrate may be, without being limited to, a chromogenic or chemiluminescent substrate specific to said enzyme.

According to the present invention, in order to assure the quality of the assay performed to measure the various parameters included in the biochemical profiles, the reference biochemical profile obtained is first compared with the predetermined reference biochemical profile provided. Providing that the reference biochemical profile obtained is identical to, i.e., of total similarity with, the predetermined reference biochemical profiles, the comparison between the test biochemical profile and either the reference biochemical profile or the predetermined reference biochemical profile can then be performed as described above.

The invention will now be illustrated by the following non-limiting Examples.

### EXAMPLES

### Experimental

### 1. Animal Experimental Design

Male Sprague-Dawley rats were treated for 14 days by daily intraperitoneal (IP) injections of haloperidol (1 mg/kg), fluvoxamine (10 mg/kg), a combination of both (1 mg/kg and 10 mg/kg, respectively) or clozapine (10 mg/kg), dissolved in a sterile saline solution containing 2% dimethylsulfoxide (DMSO) and 70 μM acetic acid. The control group included rats that were treated with the same solution without a drug. As previously demonstrated, these dosages produce levels of the drugs in rat serum which are similar to the ranges considered to be therapeutic in humans; and brain and serum concentrations of haloperidol or fluvoxamine, given individually, are not significantly changed when they are coadministered (Chertkow et al., 2007). Thus, at this dosage range, the distinctive pharmacologic profile of the combined treatment with haloperidol and fluvoxamine can not be attributed to the pharmacokinetic interaction.

The treatment period of 14 days was chosen as it is often used in chronic administration studies in animals. In addition, clinical observations show that add-on fluvoxamine caused an improvement in negative symptoms of schizophrenia (Silver et al., 2003b). An acute administration study, i.e., a single injection of the drugs, was performed in order to identify the early time-course of the drug-induced changes (30 min and 1 hr).

These studies were focused on gamma aminobutyric acid (GABA)_{A} β3 receptor subunit, glutamic acid decarboxylase 67 (GAD67) and protein kinase Cβ2 (PKCβ2), since a previous study indicated that these genes may be influenced by the combined treatment with haloperidol and fluvoxamine (Chertkow et al., 2006). Furthermore, PKC and extracellular signal-regulated kinase (ERK) expression and phosphorylation levels were assessed in order to investigate whether these proteins are involved in GABA_{A} receptor regulation by the drugs under study (Brandon et al., 2002, Bell-Horner et al., 2006). The alterations of GABA_{A}β3, GAD67, PKCβ2 and ERK1/2 proteins resulting from the administration of the various drugs were assayed by Western blotting. Phosphorylation level of PKC and ERK were determined using antibodies specific for the phosphorylated forms of these proteins.

### 1.1 Brain Dissection

For protein and RNA measurements, rats were sacrificed by decapitation, and frontal cortex, hippocampus and striatum were dissected and frozen immediately in liquid nitrogen, and then stored at −80° C. for protein analysis.

1.2 Evaluation of Protein Expression Associated with GABA System

1.2.1 Separation of whole lysate, membranal and cytosolic fractions. Brain tissue lyzate was homogenized in Tris-sucrose buffer pH 7.4 containing a mixture of protease inhibitors (Roche, Inc. and phosphatase inhibitors) and centrifugated at 1000 g for 10 min. The supernatant was kept and 100 μl of it was reserved for whole lysate protein analysis. The rest of the supernatant was centrifugated at 100,000 g for 1 hr at 4° C. to obtain a pellet consisting of the membrane fraction and a supernatant consisting of the cytosolic fraction. The pellet was resuspended in Tris-sucrose buffer, containing a mixture of protease inhibitors and 0.5% Triton X-100, placed on ice for 30 min and vortexed thoroughly. All the fractions were assayed for protein concentration by Bradford reagent.

1.2.2 Western blotting analysis. Samples containing 30 μg protein were prepared in 5× loading buffer, boiled for 10 minutes and subjected to electrophoresis through a NuPAGE 4-12% gel (Invitrogen, Groningen, The Netherlands). Upon completion of the electrophoresis, the proteins were electrophoretically transferred to a nitrocellulose membrane, and the membrane was blocked with 5% milk and immunoblotted with primary antibody and subsequently with a second, horseradish peroxidase-conjugated antibody (polyclonal anti-rabbit IgG conjugated with horseradish peroxidase, Cell Signaling, Beverly, Mass., USA; anti-mouse IgG peroxidase conjugate, Sigma, Mo., U.S.A.; or anti-goat IgG conjugated with horseradish peroxidase, Santa Cruz Biotechnology, USA). Bands were revealed by enhanced chemiluminesence using the detection reagent ECL (Amersham, Pharmacia, Little Chalfont Buckinghamshire, UK). Quantification of results was accomplished by measuring the optical density of the labeled bands from the autoradiograms, using the computerized imaging program Bio-ID (Vilber Lourmat Biotech. Bioprof, Torcy, France).

The antibodies used were affinity-purified goat anti-GABA_{A}β3 (Santa Cruz Biotechnology); mouse anti-GAD67 (BD Biosciences Pharmingen); mouse anti-PKCβ (Transduction laboratories); rabbit anti-phospho-PKC-pan (Cell Signalling); anti-ERK1/ 2, rabbit anti-phospho-ERK1/2 (Cell Signalling); and mouse anti-β-actin (Sigma, Mo., U.S.A.).

### 2. Cell Culture Experiments

Since there is, currently, no established cell line expressing dopaminergic, GABAergic and serotonergic system elements (Hales and Tyndale, 1994; Tyndale et al., 1994), primary cultures were the best option available for in vitro studies of the molecular mechanism of antipsychotic and antidepressant drugs. The major advantages of using primary cell cultures are the possibility to examine the complex influence of psychoactive medications on various neurotransmitter systems, while using desired modifications of the studied pathways and applying molecular biology techniques.

The main goal of our in vitro study was to learn more about the molecular signaling pathways which may be associated with the findings in the in vivo studies. Previous studies demonstrated the involvement of cell signal transduction pathway including PKC, protein kinase A (PKA) and mitogen-activated protein kinase (MAPK) cascade in modulation of GABA_{A} receptor phosphorylation (Mcdonald and Moss, 1997; Brandon et al., 2000; Bell-Horner et al., 2006). Therefore, we examined the modulation of GABA_{A}β subunit phosphorylation induced by the treatment with the various drugs, using specific inhibitors of these pathways, e.g., GF109203X (a non-selective PKC inhibitor), H89 (an inhibitor of phorbol-12-myristate-13-acetate, PMA, a well established PKC activator) and PD98059 (a non-selective ERK1/2 inhibitor).

### 2.1 Primary Neuronal Cortical Culture Preparation

Cerebral cortices were isolated from Sprague-Dawley rat embryonic (E18) pups, placed in petri dishes containing ice-cold Hank's Buffered Salt Solution (HBSS) and cleaned from the meninges. The cortices were then incubated in 0.25% trypsin-EDTA solution at 37° C. for 10 min. Trypsin inhibitor and DNase solution were added to terminate the trypsin activity, and mechanical dissociation was performed by intensive titration until the suspension was homogenous. Then, the cells were filtered through a 70-μm nylon cell strainer (BD Biosciences, San Jose, Calif., USA), resuspended in Minimum Essential Medium (MEM) supplemented with 10% fetal calf serum (FCS) and 100 u/ml penicillin/streptomycin solutions, and plated in culture dishes coated with poly-D-lysine. Four hours later, the medium was replaced with neurobasal medium supplemented with B27, 2 mM L-glutamine, 5 mM HEPES and 100 u/ml penicillin/streptomycin solutions. This conditions support neuronal differentiation and growth providing primarily neuronal culture (Brewer, 1995). Immunocytochemistry with monoclonal anti-MAP2 primary antibody (neuronal marker) was performed as described hereinbelow, in order to establish the integrity of the culture. Cultures were maintained in an incubator at 37° C. and 5% CO₂, and neurons were cultured in vitro for 7-8 days before the beginning of the experiments.

### 2.2 Cell Viability Assay

In order to determine the working concentration of the drugs, cell survival evaluation using MTT (3-(4,5-dimethylthiazol-2-yl)-2,5-diphenyl tetrazolium bromide; Sigma) assay was performed. After 7 days in culture, cells were placed in 96 microtiter plates at a density of 10⁴ cells/well and were treated with 0.5-100 μM of haloperidol, fluvoxamine, a combination of both or clozapine, dissolved in a sterile saline solution containing 2% dimethylsulfoxide (DMSO) and 70 μM acetic acid. Cells treated with the same solution without a drug were used the control. Haloperidol, fluvoxamine and clozapine were demonstrated not to have a toxic effect on the cell cultures at concentration of <10 μM. Since the dose of haloperidol was 10 fold lower than the dose of fluvoxamine in the in vivo studies, both alone and in the haloperidol-fluvoxamine combination, the same ratio of concentrations were chosen for the in vitro studies.

Seven days after the onset of drug treatment, the cells were incubated at dark with MTT (0.5 mg/ml) at 37° C. for 2 hrs. Finally, 10% sodium dodecyl sulfate (SDS) in 0.01 M HCl was added to dissolve the formazan product. The absorption was determined in a Tecan Sunrise Eliza-Reader (Switzerland) at λ=570 nm 24 hrs later, and the background readings at 650 nm were automatically subtracted. The results were expressed as percentage of the untreated control.

### 2.3 Preparation of Cell Lysates

In order to collect whole cell lysate after the appropriate treatment, medium was removed and 250 μl of cold lysis buffer consisting of 200 mM Tris-HCl, pH 8.0, 150 mM NaCl, 5 mM EDTA, 1 mM sodium vanadate, 1% Triton X-100, provided with protease and phosphatase inhibitor cocktails were added, and the cells were then left on ice for 10 min until solubilization. After centrifugation at 14,000 g for 10 min at 4° C., protein concentration in the supernatant was determined by Bradford reagent (Sigma, USA).

2.4 Preparation of Cytosolic and Membrane Extracts from Cell Lysates

Cells were cultured for 7 days and were then treated with the drugs for the desirable period of time. The medium was then removed and the cells were washed 3 times in ice-cold Tris-sucrose buffer and collected by scrapping in 200 μl of homogenization buffer (20 mM Tris-HCl, pH 7.4, 0.32 M sucrose, 2 mM EDTA, 50 mM β-mercaptoethanol) containing a mixture of protease inhibitors (Roche, Inc.) and phosphatase inhibitors. The mixture was sonicated twice, 10 sec each time, and centrifugated at 100,000 g for 30 hrs at 4° C. to obtain a pellet consisting of the membrane fraction and a supernatant consisting of the cytosolic fraction. The pellet was resuspended in Tris-sucrose buffer containing a mixture of protease inhibitors and 0.5% Triton X-100, placed on ice for 30 min and sonicated twice, 10 sec each time. All the fractions were assayed for protein concentration by Bradford reagent.

### 2.5 GABA_{A} Phosphoserine Immunoprecipitation Analysis

Cells were collected in lysis buffer as described above. Protein concentration was measured by Bradford assay, all the samples were diluted to the concentration of 1 μg/μl, and 250 μl of the lysate were taken for immunoprecipitation. Three μl of mouse anti-GABA_{A}β3/β2 (Upstate, Lake Placid, N.Y.) antibody were added to the lysate, following by overnight incubation at 4° C., and 50 μl of protein G sepharose were then added. The mixture was incubated for 2 hrs and centrifugated at 12,000 g for 20 sec at 4° C., and the supernatant was removed. The pellet containing receptor-antibody-bead complex was washed three times with PBS, and the receptor-antibody-bead complex was then resuspended in 50 μl loading buffer and boiled for 5 min. Beads were separated from the immunoprecipitate, which was then subjected to SDS-PAGE gel electrophoresis and analyzed by Western blotting with rabbit anti-phosphoserine antibodies (dilution of 1:500, Chemicon, Temecula, Calif.). The values obtained by densitometrical analysis were normalized to GABA_{A}β3/β2 intensity levels.

### 2.6 Immunocytochemical Assessments

Immunocytochemical experiments were performed to establish the integrity of the culture. The cells were fixed in paraformaldehyde in PBS containing 4% sucrose at 25° C. for 30 min, washed three times with PBS and then permeabilized on ice with Triton X-100 (0.1%) in sodium citrate (0.1%) for 2 min. After three rinses with PBS, cells were incubated in PBS containing 10% donkey serum at 37° C. for 1 hr to block non-specific staining. Cultures were incubated at 4° C. overnight with monoclonal anti-MAP2 primary antibody (neuronal marker) diluted in PBS containing 1% donkey serum and 0.05% Triton X-100. Wells were aspirated and rinsed twice with PBS for 10 min before the addition of anti-mouse IgG-fluorescein-conjugated antibody diluted in PBS, 1% donkey serum and 0.05% Triton X-100. After incubation at room temperature in dark for 1 hr, wells were rinsed three times in PBS and coverslips were mounted with Vectashield (Vector Laboratories, Burlingame, Calif., USA). Immunofluorescence was observed using a 60×objective (NA 1.4) and Radiance 2000 confocal system (Bio-Rad, Hercules, Calif., USA) supported with Laser-Sharp 2000 software.

### Example 1

### Haloperidol, Fluvoxamine and Clozapine Alter GABA_{A}β3, PKCβ, PKCγ and Bad mRNA Levels in Frontal Cortices of Rats

In this experiment we examined the molecular alterations in frontal cortices of rats chronically-treated with the haloperidol-fluvoxamine combination vs. each one of these drugs or clozapine. In particular, male Sprague-Dawley rats were treated for 14 days by daily intraperitoneal (IP) injections of haloperidol, fluvoxamine, the haloperidol-fluvoxamine combination, clozapine or vehicle, as described in Materials and Methods. cDNA array was used to generate a list of candidate genes that may play a role in the mechanism of action of antipsychotic treatment, in particular, of the haloperidol-fluvoxamine combination.

From the list of genes altered (data not shown), eight were chosen for further verification by real-time RT-PCR and for examination of the corresponding expression at the protein level. These genes included cyclin D3, encoding for a regulator of cell proliferation; the C-family protein kinases PKCγ and PKCβ, that modulates various cellular processes; Bad, which codes for a pro-apoptotic protein; Presenilin 1, involved in processing amyloid precursor protein, thereby playing a role in Alzheimer brain disease; the gamma-aminobutyric acid (GABA) related genes: glutamic acid decarboxylase 67 (GAD67) and GABA_{A}β3; and the regulator of G protein signaling 4 (RGS4), encoding for one protein of a large family controlling the activity of G-protein coupled receptors.

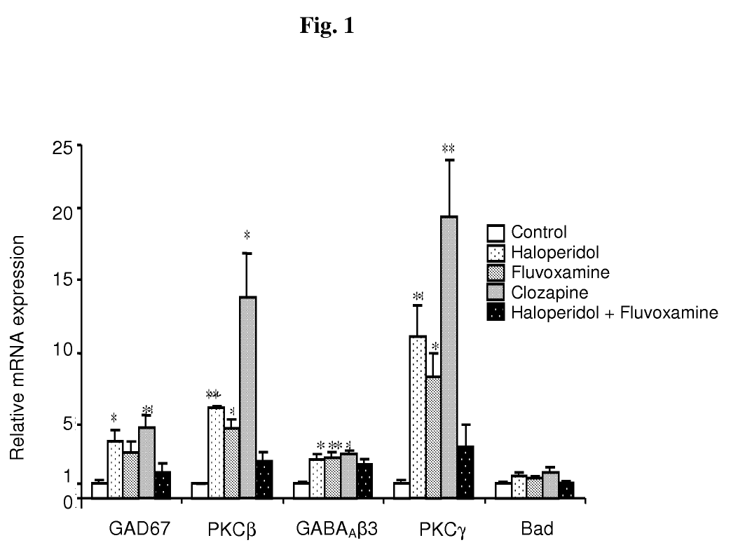

The mRNA levels of genes selected based on the array results were quantified by real-time RT-PCR, and their relative mRNA expression is shown in FIG. 1. The mRNA concentration of the pro-apoptotic gene, Bad, was not affected by any of the drugs. Haloperidol and clozapine significantly increased the mRNA levels of the GABAergic genes GAD67 and GABA_{A}β3, and also of the PKC family genes PKCβ and PKCγ. PKCβ, GABA_{A}β3 and PKCγ were increased significantly also by fluvoxamine. The haloperidol-fluvoxamine combination had no statistically significant effect on any of these genes.

### Example 2

### The Haloperidol-Fluvoxamine Combination and Clozapine Decrease GABA_{A}β3 Protein Level and Induce Receptor Endocytosis in Frontal Cortices of Rats

Previous studies demonstrated that the expression levels of various proteins associated with the GABA system in the frontal cortex are affected by chronic treatment with the drugs of interest. Thus, in this experiment we examined the expression level of GABA_{A}β3 receptor subunit in the frontal cortices of rats chronically-treated with haloperidol, fluvoxamine, the haloperidol-fluvoxamine combination or clozapine. Protein samples from individual frontal cortices were subjected to subcellular fractionation and consequent Western blot analysis using primary antibodies against GABA_{A}β3. Immunoreactive bands were analyzed by densitometry and normalized against β-actin levels.

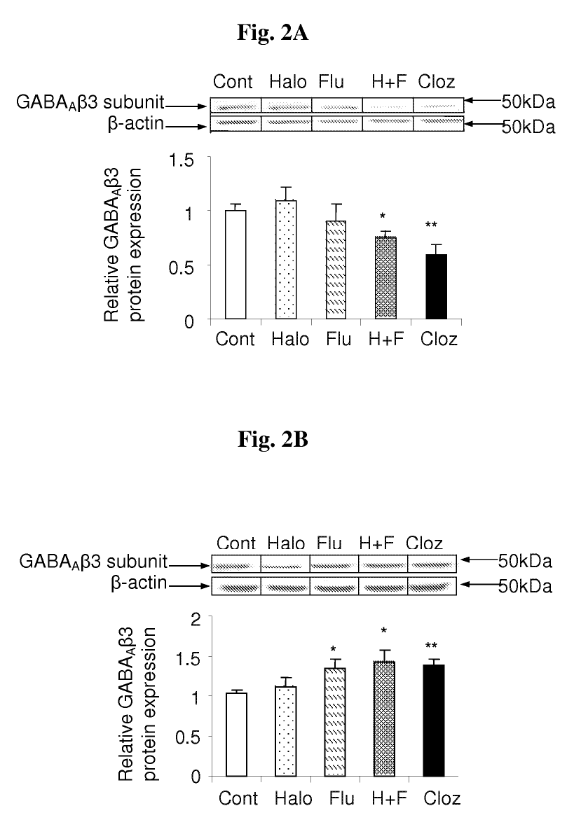

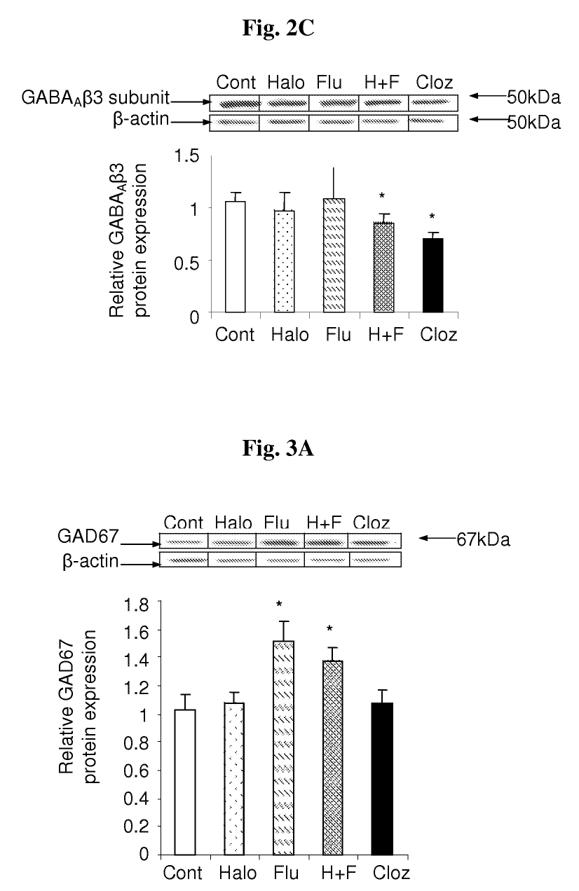

As shown in FIGS. 2A-2C, both the haloperidol-fluvoxamine combination and clozapine significantly decreased relative GABA_{A}β3 receptor subunit expression level in rat frontal cortices, while each individual drug did not induce such an effect (2A). Furthermore, both the haloperidol-fluvoxamine combination and clozapine induced GABA_{A}β3 receptor translocation from the membranal to the cytosolic compartment, i.e., increased the level of the GABA_{A}β3 receptor subunit in the cytosolic compartment (2B) and decreased its level in the membranal compartment (2C).

### Example 3

### The Effect of Haloperidol, Fluvoxamine, the Haloperidol-Fluvoxamine Combination and Clozapine on GAD67, PKCβ2 and ERK1/2 Protein Levels in Frontal Cortices of Rats

In this experiment we examined the expression levels of (i) GAD67, the key enzyme in GABA synthesis; (ii) PKCβ2, a PKC isoform that regulates GABA_{A}β3 subunit phosphorylation; and (iii) both ERK1 (p44-MAPK) and ERK2 (p42-MAPK), previously shown to affect GABA_{A} receptor activity, in the frontal cortices of rats chronically-treated with haloperidol, fluvoxamine, the haloperidol-fluvoxamine combination or clozapine. Whole tissue lysates from individual frontal cortices were subjected to Western blot analysis using primary antibodies. Immunoreactive bands were analyzed by densitometry and normalized against β-actinlevels.

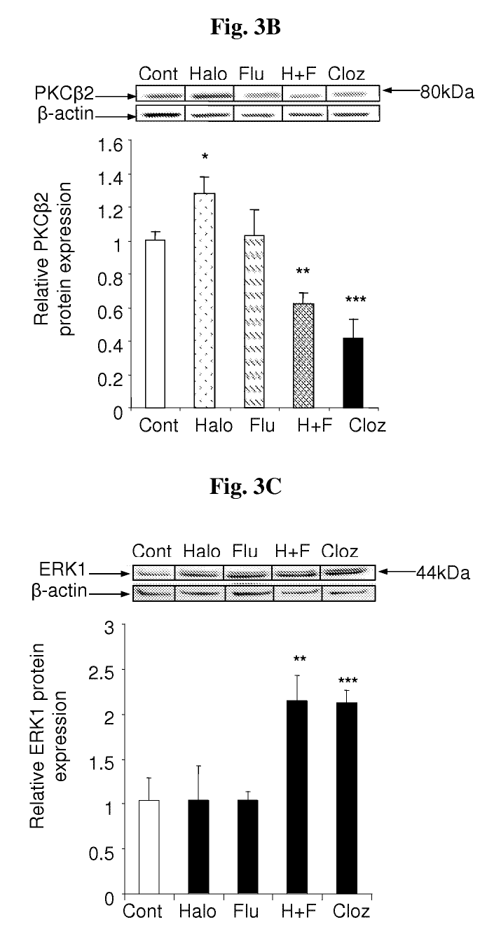

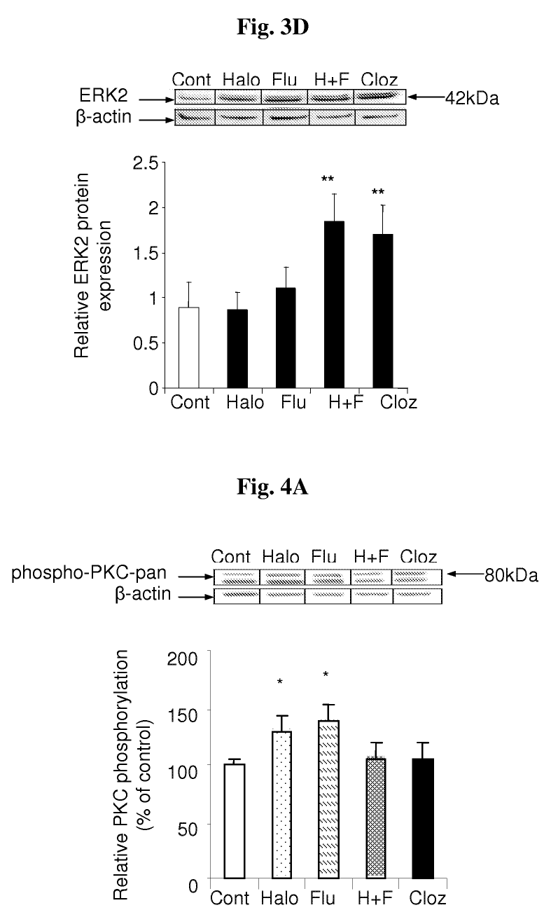

FIG. 3A shows that both fluvoxamine and the haloperidol-fluvoxamine combination increased the relative GAD67 expression level in rat frontal cortices. It is concluded that this effect is due to the serotonergic action of fluvoxamine, since no additive effect was observed in the haloperidol-fluvoxamine-treated group compared with the fluvoxamine-treated group. FIG. 3B shows that PKCβ2 protein level in haloperidol-treated rats were significantly increased compared to controls, while both the haloperidol-fluvoxamine combination and clozapine significantly decreased PKCβ2 protein expression. FIGS. 3C-3D show that both ERK1 and ERK2 protein levels were significantly increased in animals administrated with either the haloperidol-fluvoxamine combination or clozapine.

### Example 4

### The Haloperidol-Fluvoxamine Combination and Clozapine do not Affect PKC Phosphorylation in Frontal Cortices of Rats

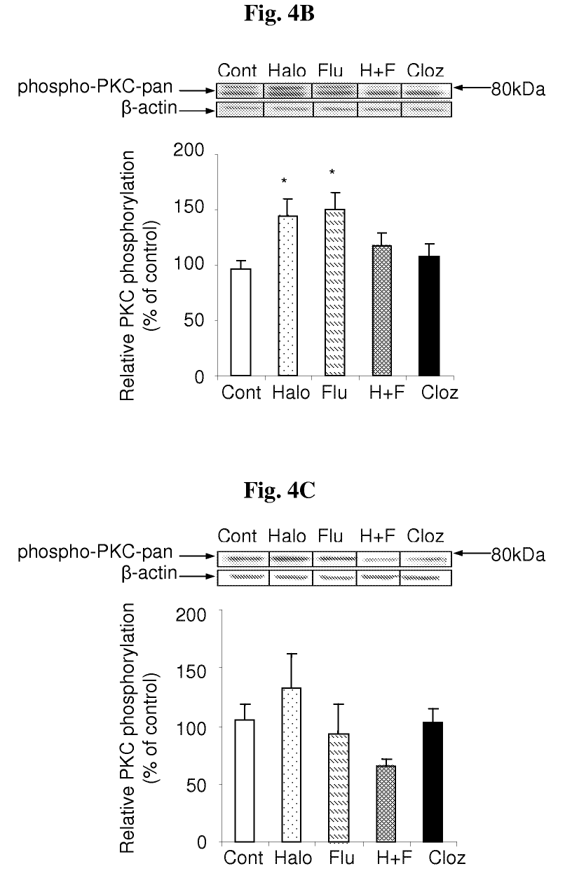

In order to investigate possible involvement of PKC in the mechanism of action of the various drugs, the total PKC phosphorylation level was examined using anti-phospho-PKC-pan antibodies. Protein samples from individual frontal cortices of rats chronically-treated with haloperidol, fluvoxamine, the haloperidol-fluvoxamine combination or clozapine were subjected to subcellular fractionation and consequent Western blot analysis of the whole lysate, cytosolic or membranal compartment, using primary antibodies against phospho-PKC-pan. Immunoreactive bands were analyzed by densitometry and normalized against β-actin levels. As shown in FIGS. 4A-4C, the individual drug treatment with either haloperidol or fluvoxamine increased PKC phosphorylation in whole lysate (38% and 40%, respectively) (4A) and in the membranal fraction (45% and 50%, respectively) (4B), while the haloperidol-fluvoxamine combination or clozapine did not affect PKC phosphorylation. None of the drug treatments were found to affect the activation of PKC in the cytosolic fraction (4C). In view of the fact that activation of PKC by phosphorylation increases PKC translocation to membranal compartment, these results indicate that individual treatment with either haloperidol or fluvoxamine upregulates PKC activation; however, this effect is abolished while these drugs are co-administrated.

### Example 5

### The Haloperidol-Fluvoxamine Combination and Clozapine Decrease ERK1/2 Phosphorylation in Frontal Cortices of Rats

The phosphorylation of the important protein of mitogen-activated protein kinases (MAPK) pathway, ERK1/2, was determined using anti-phospho-ERK1/2 antibodies, which specifically bind to the phosphorylated form of ERK1 and ERK2. Whole tissue lysates of frontal cortices of rats chronically-treated with haloperidol, fluvoxamine, the haloperidol-fluvoxamine combination or clozapine were subjected to Western blot analysis using primary antibodies. Immunoreactive bands were analyzed by densitometry and normalized against ERK1 and ERK2 levels.

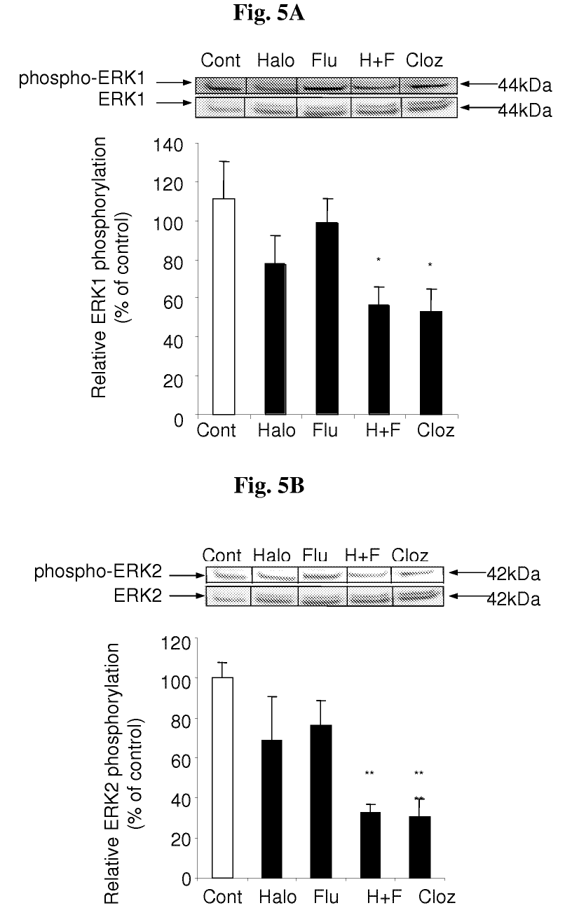

As shown in FIGS. 5A-5B, relative phosphorylation levels in whole lysate were significantly decreased by both the haloperidol-fluvoxamine combination and clozapine. As GABA_{A} was previously demonstrated to be phosphorylated by ERK and, vise versa, GABA_{A} was described to mediate ERK phosphorylation, the differential regulation of MAPK pathway shown in the present study might be associated with the observed alterations in GABAergic system elements induced by the certain drug treatments described above.

### Example 6

### The Effects of Acute Administration of Haloperidol-Fluvoxamine Combination and Clozapine on Cytosolic and Membranal GABA_{A}β3 and PKCβ2 Protein Levels, and on ERK2 Phosphorylation Level in Frontal Cortices of Rats

In this experiment we examined the expression level of GABA_{A}β3 receptor subunit and PKCβ2, as well as the ERK2 phosphorylation level in cytosolic and membranal compartments of frontal cortices of rats administered with a single intraperitoneal (IP) injection (acute treatment) of haloperidol, fluvoxamine, the haloperidol-fluvoxamine combination or clozapine. In particular, rats were administered with haloperidol, fluvoxamine, the haloperidol-fluvoxamine combination, clozapine or vehicle, and were sacrificed 30 minutes or 1 hr later. Protein samples from individual frontal cortices were subjected to subcellular fractionation and consequent Western blot analysis of the cytosolic or membranal compartments, using primary antibodies. Immunoreactive bands were analyzed by densitometry and normalized against β-actin levels in the cases of GABA_{A}β3 receptor subunit and PKCβ2, and against total ERK2 levels in the case of ERK2 phosphorylation level.

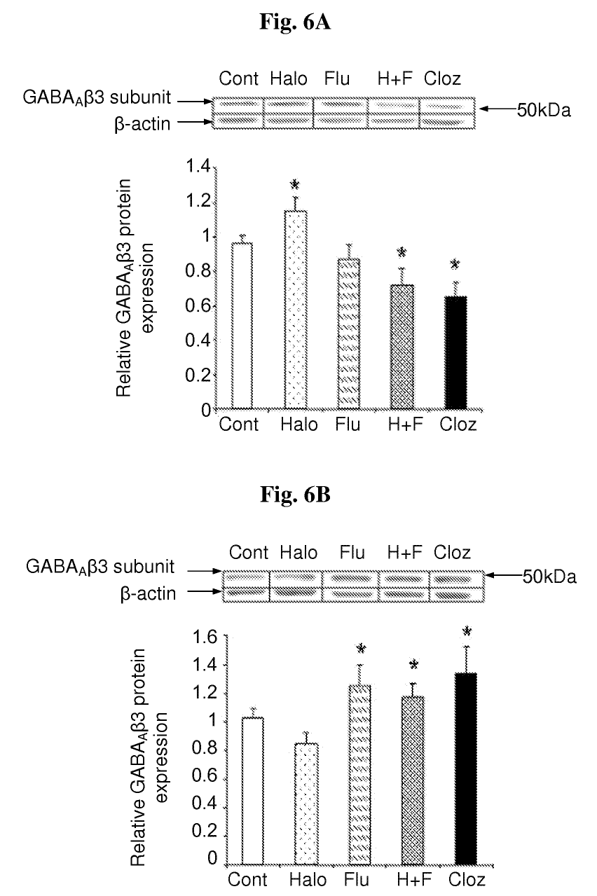

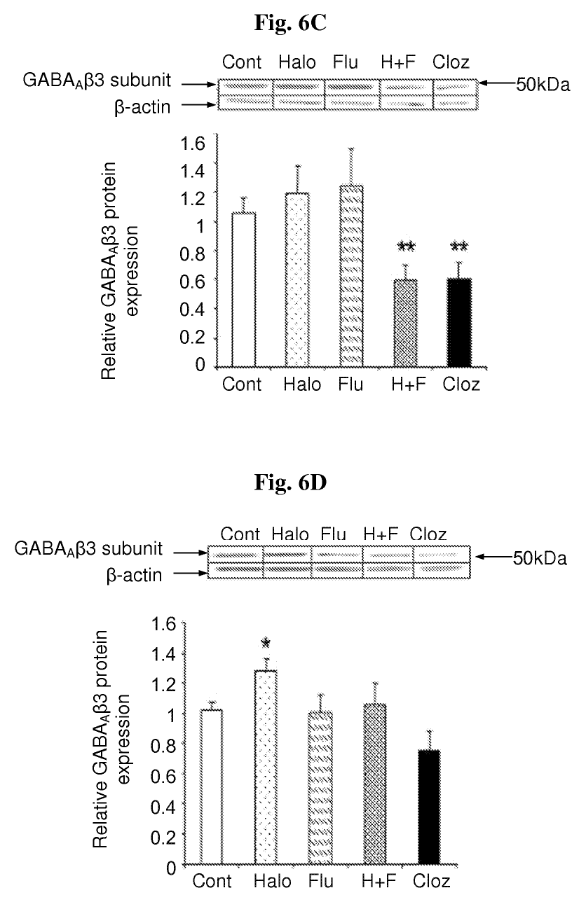

As shown in FIGS. 6A-6D, whereas chronic treatment with the haloperidol-fluvoxamine combination and clozapine induced GABA_{A}β3 receptor internalization (see Example 2 above), acute treatment with the haloperidol-fluvoxamine combination and clozapine decreased the level of the GABA_{A}β3 receptor subunit in the cytosolic compartment and increased its level in the membranal compartment 30 minutes following injection. One hour following the injection, a reduced level of GABA_{A}β3 receptor subunit was still measured in the cytosolic compartment; however, no significant changes in GABA_{A}β3 receptor subunit levels were measured in the membranal compartment. The differences between the GABA_{A}β3 receptor subunit levels measured in the cytosolic and membranal compartments following acute vs. chronic treatments may be due to the time dependent-effect and -regulation of GABA receptor levels induced by the treatment with the haloperidol-fluvoxamine combination or clozapine.

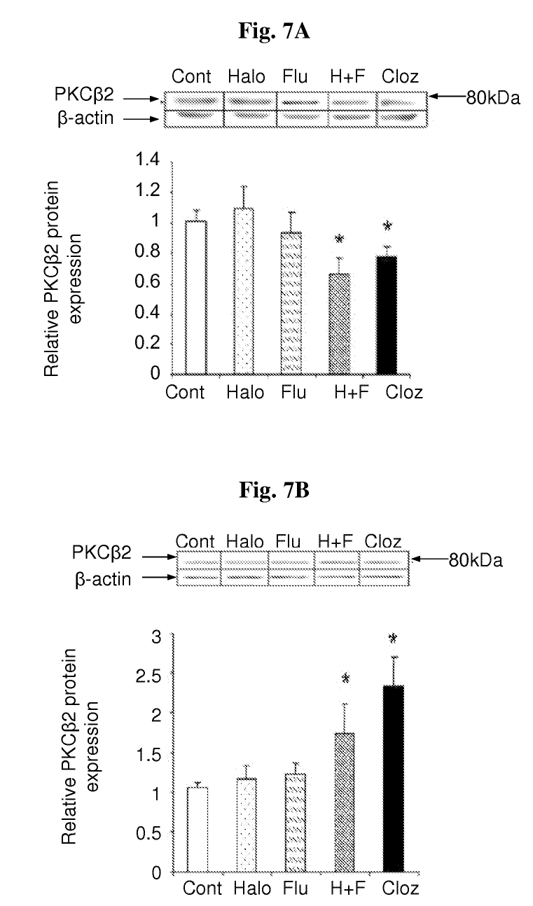

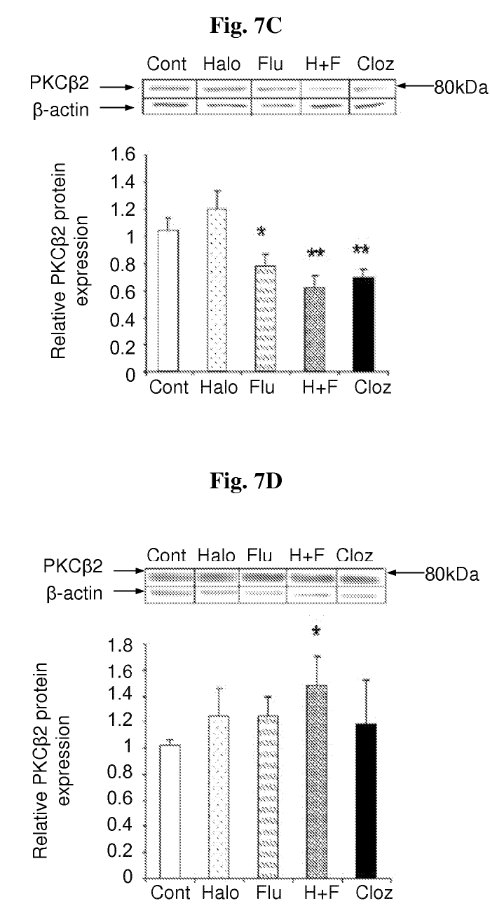

As shown in FIGS. 7A-7D, acute treatment with the haloperidol-fluvoxamine combination and clozapine decreased the level of the PKCβ2 subunit in the cytosolic compartment and increased its level in the membranal compartment 30 minutes following injection. One hour following that treatment, as well as following an acute treatment with fluvoxamine, a reduced level of PKCβ2 subunit was still measured in the cytosolic compartment; however, a significant change (increase) in PKCβ2 subunit level in the membranal compartment was measured only in rats treated with the haloperidol-fluvoxamine combination. The similarity between the results observed for GABA_{A}β3 receptor and PKCβ2 levels following acute treatment may suggest an association between GABAergic system elements and PKC signaling pathway regulated by the certain drug treatments described above.

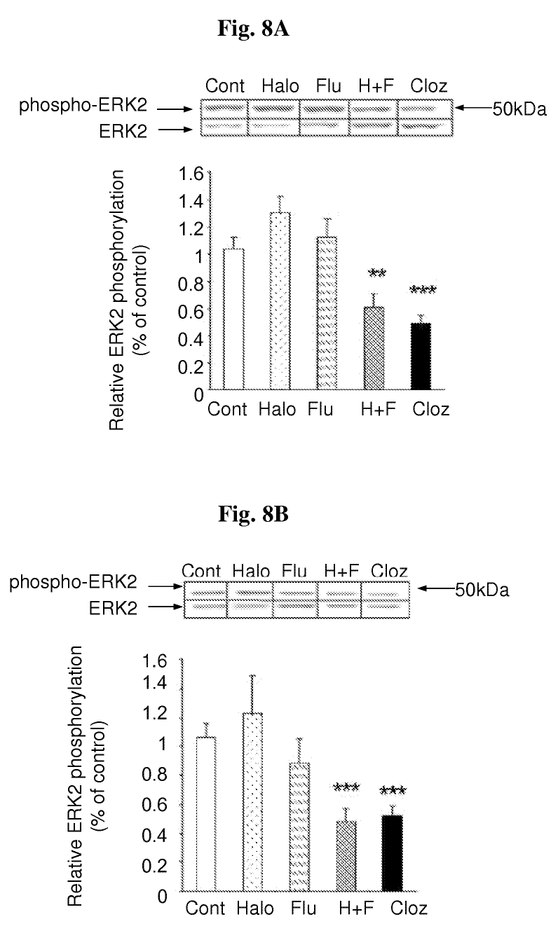

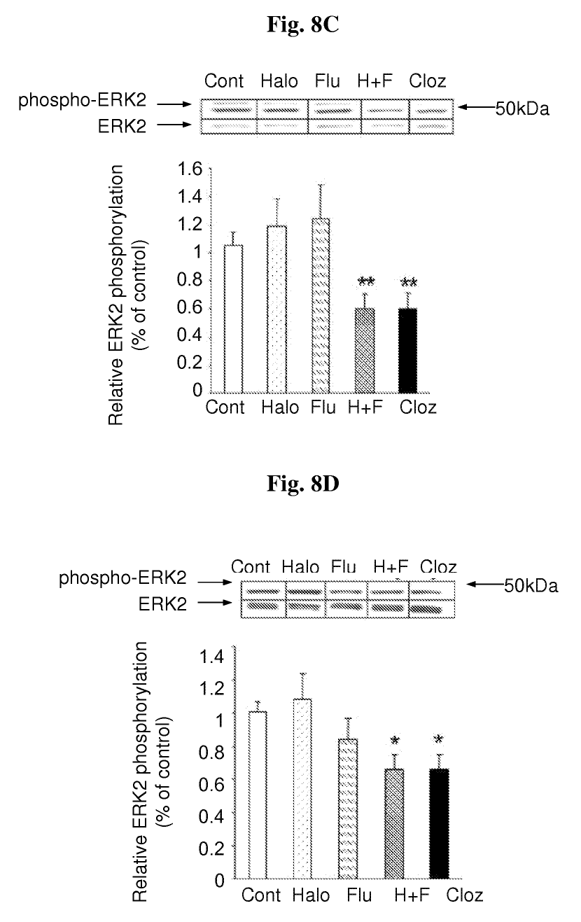

As shown in FIGS. 8A-8D, acute treatment with the haloperidol-fluvoxamine combination and clozapine decreased the level of the phosphorylated ERK2 subunit both in the cytosolic and membranal compartments 30 minutes and 1 h following injection, as observed following chronic administration of these treatments as well (FIG. 5B). These data indicate that MAPK pathway might be associated with the observed alterations in GABAergic system elements induced by the certain drug treatments described above.

### Example 7

### The Haloperidol-Fluvoxamine Combination and Clozapine Increase GABA_{A}β2/β3 Phosphorylation in vitro

In order to investigate the possible phosphorylative mechanism which might be responsible for GABA_{A} receptor internalization induced by both the haloperidol-fluvoxamine combination and clozapine, as shown in Example 2 above, we have treated rat primary cortical neuronal cell cultures with haloperidol, fluvoxamine, the haloperidol-fluvoxamine combination or clozapine for 15 minutes (acute treatment) or 7 days (chronic treatment), as described in Materials and Methods. Immunoprecipitation with anti-GABA_{A}β2/β3 antibodies was performed, and the obtained lysates were subjected to Western blot analysis using primary antibodies against phospho-serine. Immunoreactive bands were analyzed by densitometry and normalized against total GABA_{A}β2/β3 levels.

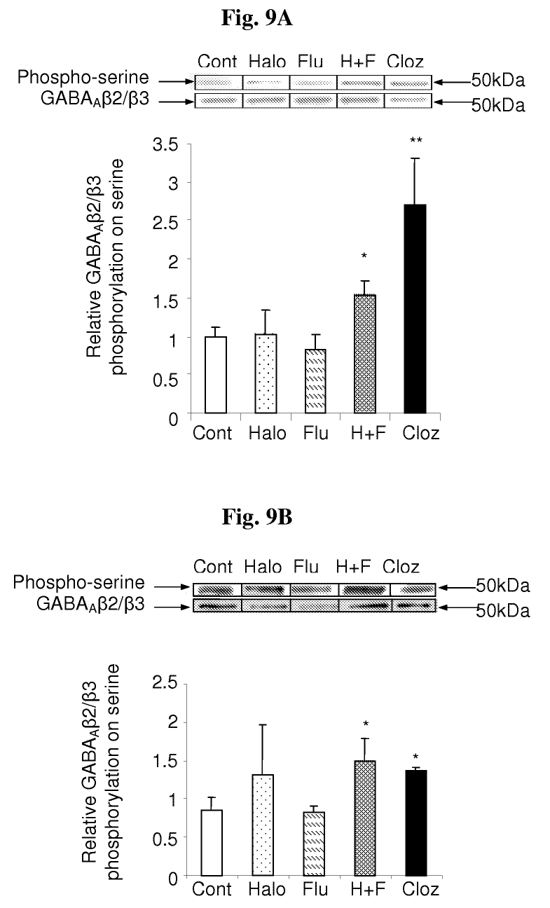

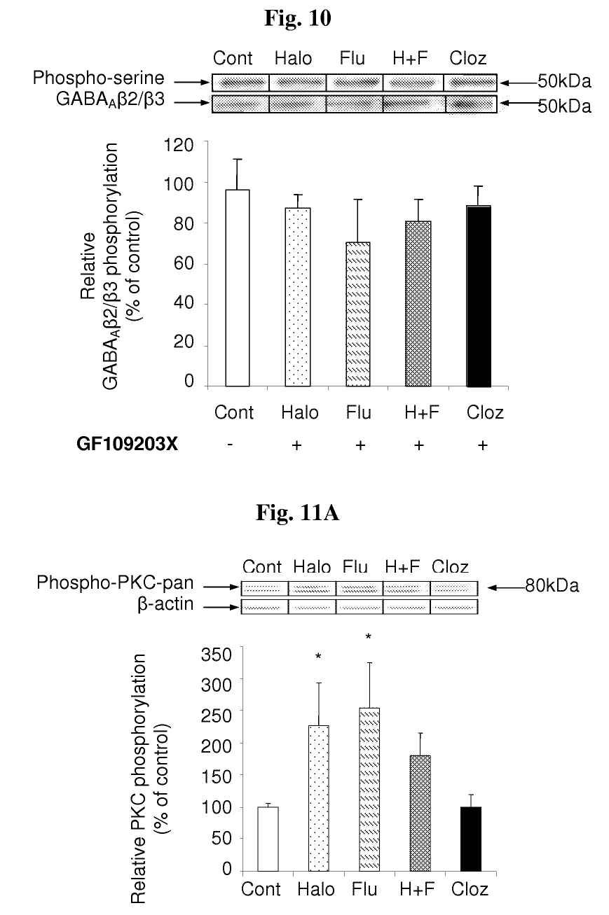

As shown in FIG. 9A, GABA_{A}β2/β3 subunit phosphorylation was significantly increased compared to controls following the acute treatment with both the haloperidol-fluvoxamine combination (52%) and clozapine (169%); and a similar effect was obtained following the corresponding chronic treatments, increasing the GABA_{A}β2/β3 phosphorylation by 49% and 37%, respectively, as shown in FIG. 9B. The treatment with either haloperidol or fluvoxamine alone did not alter GABA_{A}β2/β3 phosphorylation.

### Example 8

### PKC Inhibition Prevents the Effect of the Haloperidol-Fluvoxamine Combination and clozapine on GABA_{A}β2/β3 Phosphorylation in vitro

In order to investigate the involvement of PKC in the effect induced by the haloperidol-fluvoxamine combination and clozapine on GABA_{A}β2/β3 phosphorylation, as shown in Example 7 above, primary cortical neuronal cell cultures were pretreated for 30 minutes with the non-selective PKC inhibitor GF109203X, and were then incubated for 15 minutes with haloperidol, fluvoxamine, the haloperidol-fluvoxamine combination, clozapine or vehicle, as described in Materials and Methods. Immunoprecipitation with anti-GABA_{A}β2/β3 antibodies was performed, and the obtained lysates were subjected to Western blot analysis using primary antibodies against phospho-serine. Immunoreactive bands were analyzed by densitometry and normalized against total GABA_{A}β2/β3 levels.

As shown in FIG. 10, the pre-treatment with GF109203X prevented the effect induced by both the haloperidol-fluvoxamine combination and clozapine on GABA_{A}β2/β3 subunit phosphorylation, indicating that PKC pathway, which is involved in the mechanism of action of both the haloperidol-fluvoxamine combination and clozapine, may be crucial for GABA_{A}β2/β3 phosphorylation induced by these drugs.

### Example 9

### The Effects of Haloperidol, Fluvoxamine, the Haloperidol-Fluvoxamine Combination and Clozapine on PKC Protein Level and Activation in vitro

In this experiment we measured the PKC phosphorylation level in primary cortical neuronal cell cultures administered with the various drugs. In particular, primary cortical neuronal cell cultures were treated for 15 minutes (acute treatment) or 7 days (chronic treatment) with haloperidol, fluvoxamine, the haloperidol-fluvoxamine combination, clozapine or vehicle, as described in Materials and Methods. Whole cell lysates were subjected to Western blot analysis using primary antibodies against phospho-PKC-pan or PKCβ2. Immunoreactive bands were analyzed by densitometry and normalized against β-actin levels.

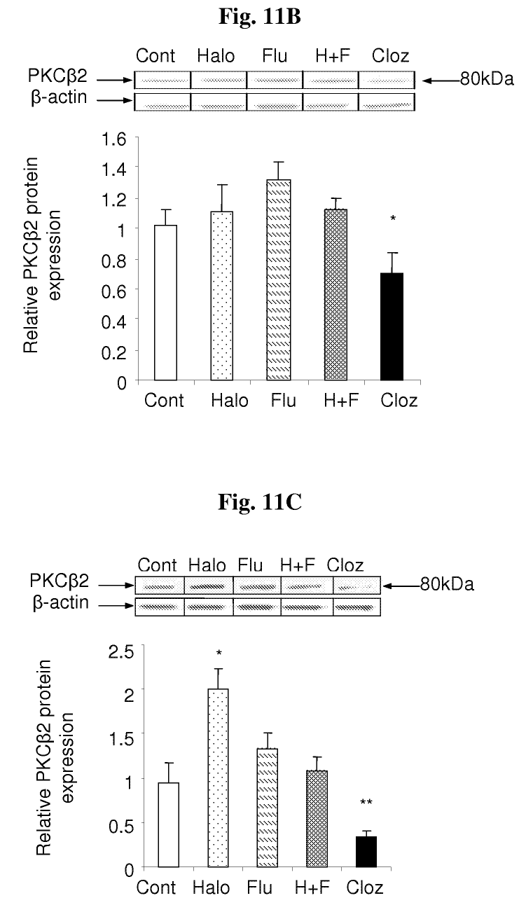

As found in the in vivo experiment described in Example 4 above, and shown in FIG. 11A, PKC phosphorylation level was significantly increased by both haloperidol (134%) and fluvoxamine (156%), while the haloperidol-fluvoxamine combination and clozapine failed to produce any significant effect. Furthermore, PKCβ2 protein level was significantly decreased by both acute (31%) and chronic (60%) treatments with clozapine, as shown in FIGS. 11B and 11C, respectively. Chronic treatment with haloperidol significantly increased PKCβ2 protein level (106%), while fluvoxamine or the haloperidol-fluvoxamine combination did not affect PKCβ2 protein levels.

### Example 10

### The Effect of the Haloperidol-Fluvoxamine Combination and Clozapine on GABA_{A}β2/β3 Phosphorylation in vitro does not Require MAPK Activation

In order to investigate the MAPK pathway involvement in the effect induced by the haloperidol-fluvoxamine combination and clozapine on GABA_{A}β2/β3 phosphorylation, as shown in Example 7 above, primary cortical neuronal cell cultures were preterated for 30 minutes with the non-selective ERK1/2 inhibitor PD98059, and were then incubated for 15 minutes with haloperidol, fluvoxamine, the haloperidol-fluvoxamine combination, clozapine or vehicle, as described in Materials and Methods. Immunoprecipitation with anti-GABA_{A}β2/β3 antibodies was performed and the obtained lysates were subjected to Western blot analysis using primary antibodies against phospho-serine. Immunoreactive bands were analyzed by densitometry and normalized against total GABA_{A}β2/β3 levels.

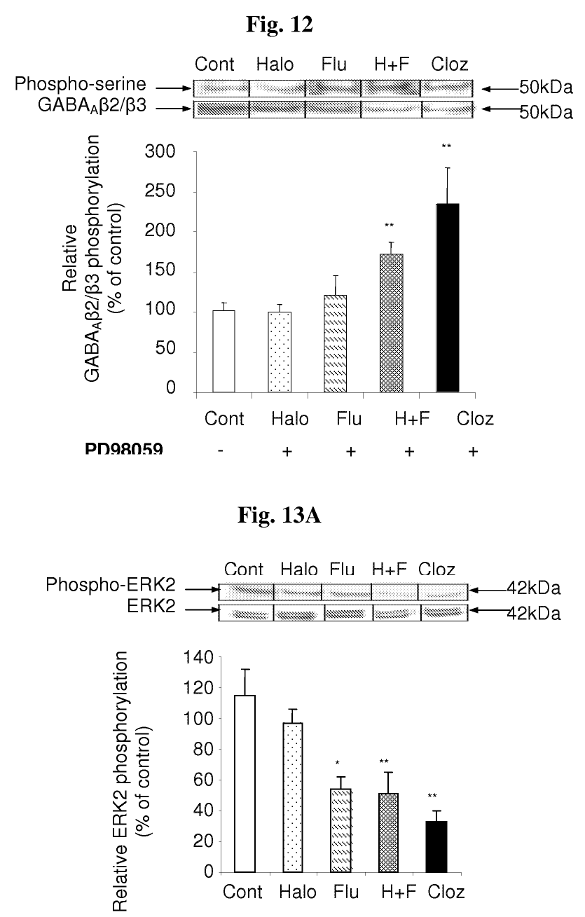

As shown in FIG. 12, the increases of GABA_{A}β2/β3 subunit phosphorylation following the treatment with the haloperidol-fluvoxamine combination (70%) and clozapine (130%) remained unaltered following the ERK inhibition, indicating that ERK is not involved in the modulation of GABA_{A}β2/β3 phosphorylation induced by the haloperidol-fluvoxamine combination or clozapine.

### Example 11

### Fluvoxamine, the Haloperidol-Fluvoxamine Combination and Clozapine alter ERK1/2 Phosphorylation in vitro

Since chronic treatments with the haloperidol-fluvoxamine combination or clozapine were found to regulate ERK1/2 phosphorylation, as shown in Example 5 above, in this experiment we examined whether said treatments modulate the activation of ERK1/2 isoforms, using specific antibodies against phosphorylated forms of ERK1/2. In particular, primary cortical neuronal cell cultures were treated for 15 minutes (acute treatment) or 7 days (chronic treatment) with haloperidol, fluvoxamine, the haloperidol-fluvoxamine combination, clozapine or vehicle, as described in Materials and Methods. Whole cell lysates were subjected to Western blot analysis using primary antibodies against phospho-ERK1/2 or ERK1/2. Immunoreactive bands were analyzed by densitometry and normalized against total ERK2 or β-actin levels.

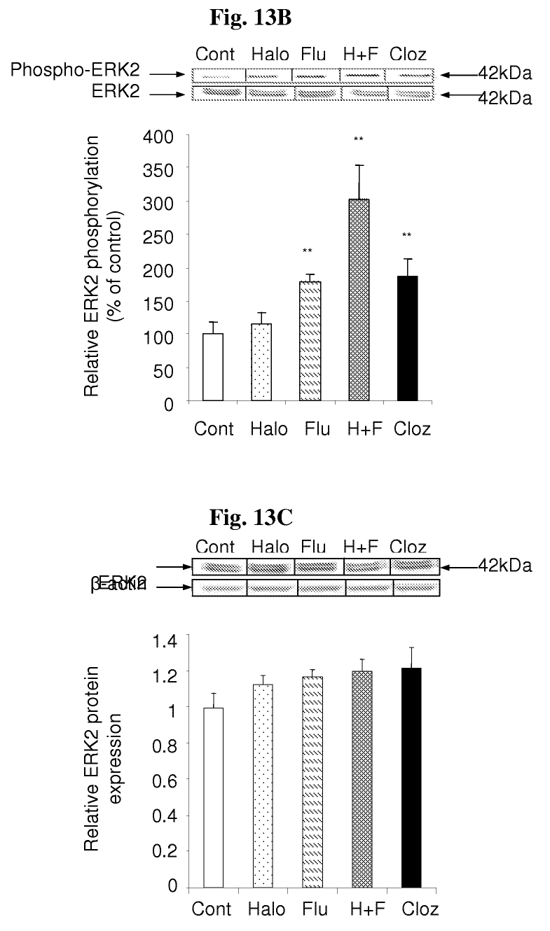

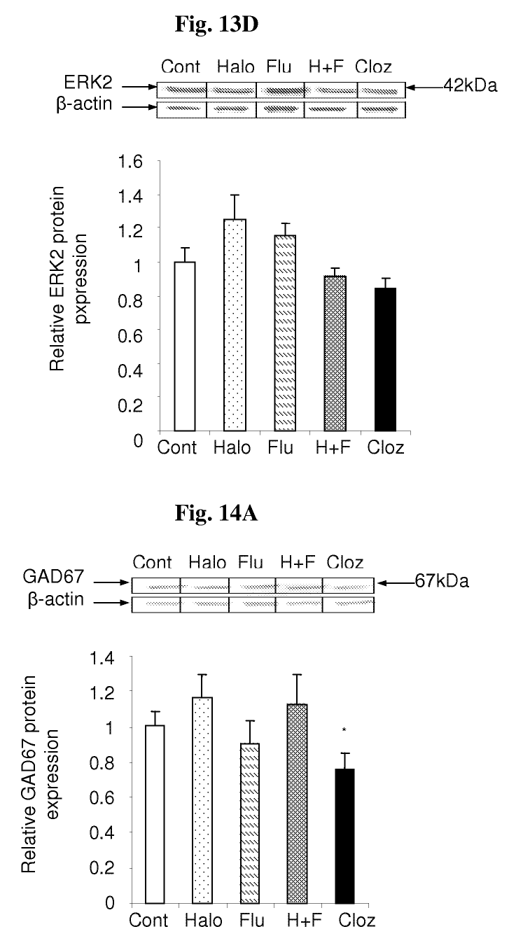

As shown in FIGS. 13A-13B, whereas haloperidol did not alter ERK2 phosphorylation in neither acute nor chronic treatment regime, fluvoxamine, the haloperidol-fluvoxamine combination and clozapine significantly altered ERK2 phosphorylation. In particular, acute treatments with fluvoxamine, the haloperidol-fluvoxamine combination and clozapine significantly reduced ERK2 phosphorylation by 46%, 49% and 67%, respectively (FIG. 13A), whereas chronic treatments with these drugs significantly increased ERK2 phosphorylation by 78%, 200% and 87%, respectively (FIG. 13B). As shown in FIGS. 13C-13D, ERK2 protein levels were not changed with neither acute (13C) nor chronic (13D) treatments with any of these drugs.

### Example 12

### Fluvoxamine, the Haloperidol-Fluvoxamine Combination and Clozapine Reduce GAD67 Protein Level in vitro

In this experiment, primary cortical neuronal cell cultures were treated for 15 minutes (acute treatment) or 7 days (chronic treatment) with haloperidol, fluvoxamine, the haloperidol-fluvoxamine combination, clozapine or vehicle, as described in Materials and Methods. Whole cell lysates were subjected to Western blot analysis using primary antibodies against GAD67. Immunoreactive bands were analyzed by densitometry and normalized against β-actin levels.

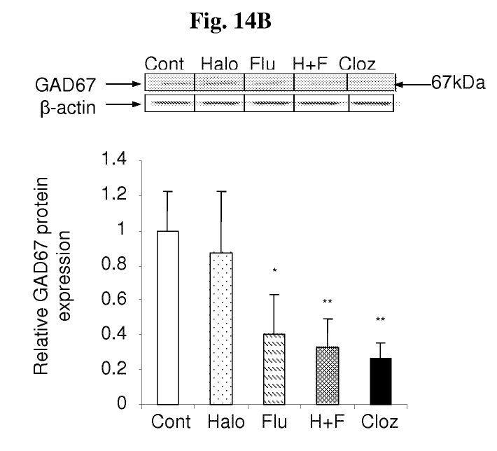

As shown in FIGS. 14A-14B, clozapine significantly decreased GAD67 protein level following both acute and chronic treatments (25% and 73%, respectively). Significant reduction in GAD67 relative protein level was also observed following chronic treatment with fluvoxamine (60%) or the haloperidol-fluvoxamine combination (67%). Haloperidol did not affect GAD67 protein level with neither acute nor chronic treatment.

### REFERENCES

Bell-Horner C. L., Dohi A., Nguyen Q., Dillon G. H., Singh M., ERK/MAPK pathway regulates GABA_{A} receptors, J Neurobiol., 2006, 66, 1467-1474
Brandon N. J., Delmas P., Kittler J. T., McDonald B. J., Sieghart W., Brown D. A., Smart T. G., Moss S. J., GABA_{A} receptor phosphorylation and functional modulation in cortical neurons by a protein kinase C-dependent pathway, J Biol. Chem., 2000, 275, 38856-38862
Brandon N. J., Jovanovic J. N., Smart T. G., Moss S. J., Receptor for activated C kinase-1 facilitates protein kinase C-dependent phosphorylation and functional modulation of GABA_{A} receptors with the activation of G-protein-coupled receptors, J Neurosci., 2002, 22, 6353-6361
Brewer G. J., Serum-free B27/neurobasal medium supports differentiated growth of neurons from the striatum, substantia nigra, septum, cerebral cortex, cerebellum, and dentate gyrus, J Neurosci Res., 1995, 42, 674-683
Cai X., Flores-Hernandez J., Feng J., Yan Z., Activity-dependent bidirectional regulation of GABA_{A} receptor channels by the 5-HT₄ receptor-mediated signalling in rat prefrontal cortical pyramidal neurons, J. Physiol., 2002, 540, 743-759
Chen G., Kittler J. T., Moss S. J., Yan Z., Dopamine D₃ receptors regulate GABA_{A} receptor function through a phospho-dependent endocytosis mechanism in nucleus accumbens, J. Neurosci., 2006, 26, 2513-2521
Chertkow Y., Weinreb O., Youdim M. B., Silver H., The effect of chronic co-administration of fluvoxamine and haloperidol compared to clozapine on the GABA system in the rat frontal cortex, Int J. Neuropsychopharmacol., 2006, 9, 287-296
Chertkow Y., Weinreb O., Youdim M. B., Silver H., Dopamine and serotonin metabolism in response to chronic administration of fluvoxamine and haloperidol combined treatment, J Neural Transm., 2007, 114, 1443-1454
Feng J., Cai X., Zhao J., Yan Z., Serotonin receptors modulate GABA_{A} receptor channels through activation of anchored protein kinase C in prefrontal cortical neurons, J. Neurosci., 2001, 21, 6502-6511
Goff D. C., Midha K. K., Sarid-Segal O., Hubbard J. W., Amico E., A placebo-controlled trial of fluoxetine added to neuroleptic in patients with schizophrenia, Psychopharmacology (Ber), 1995, 117, 417-423
Hales T. G., Tyndale R. F., Few cell lines with GABA_{A} mRNAs have functional receptors, J Neurosci., 1994, 14, 5429-5436
Lieberman J., Stroup T. S., McEvoy J., Swartz M., Rosenheck R., Perkins D. O., Keefe R. S. E., Davis S. M., Davis C. E., Lebowitz B. D., Severe J., Hsiao J. K., Effectiveness of antipsychotic drugs in patients with chronic schizophrenia, N Engl J. Med., 2005, 353, 1209-1223
McDonald B. J., Moss S. J., Conserved phosphorylation of the intracellular domains of GABA_{A} receptor β2 and β3 subunits by cAMP-dependent protein kinase, cGMP-dependent protein kinase, protein kinase C and Ca2⁺/calmodulin type II-dependent protein kinase, Neuropharmacology, 1997, 36, 1377-1385
Murphy B. P., Chung Y. C., Park T. W., McGorry P. D., Pharmacological treatment of primary negative symptoms in schizophrenia: a systematic review, Schizophr Res., 2006, 88, 5-25
Silver H., Nassar A., Fluvoxamine improves negative symptoms in treated chronic schizophrenia: an add-on double-blind, placebo-controlled study, Biol Psychiatry, 1992, 31, 698-704
Silver H., Kushnir M., Kaplan A., Fluvoxamine augmentation in clozapine-resistant schizophrenia: an open pilot study, Biol Psychiatry, 1996, 40, 671-674
Silver H., Youdim M. B., MAO-A and MAO-B activities in rat striatum, frontal cortex and liver are unaltered after long-term treatment with fluvoxamine and desipramine, Eur Neuropsychopharmacol., 2000, 10, 125-128
Silver H., Nassar A., Aharon N., Kaplan A., The onset and time course of response of negative symptoms to add-on fluvoxamine treatment, Int Clin Psychopharmacol, 2003a, 18, 87-92
Silver H., Aharon N., Kaplan A., Add-on fluvoxamine improves primary negative symptoms: evidence for specificity from response analysis of individual symptoms, Schizophr Bull., 2003b, 29, 541-546
Silver H., Shmugliakov N., Augmentation with fluvoxamine but not maprotiline improves negative symptoms in treated schizophrenia: evidence for a specific serotonergic effect from a double-blind study, J Clin Psychopharmacol., 1998, 18, 208-211

Spina E., De Domenico P., Ruello C., Longobardo N., Gitto C., Ancione M., Di Rosa A. E., Caputi A. P., Adjunctive fluoxetine in the treatment of negative symptoms in chronic schizophrenic patients, Int Clin Psychopharmacol., 1994, 9, 281-285
Tyndale R. F., Hales T. G., Olsen R. W., Tobin A. J., Distinctive patterns of GABA_{A} receptor subunit mRNAs in 13 cell lines, J Neurosci., 1994, 14, 5417-5428
van Hes R., Smid P., Stroomer C. N. J., Tipker K., Tulp M. T. M., van der Heyden J. A. M., McCreary A. C., Hesselink M. B., Kruse C. G., SLV310, a novel, potential antipsychotic, combining potent dopamine D₂ receptor antagonism with serotonin reuptake inhibition, Bioorg. Med. Chem. Lett., 2003, 13, 405-408
Wang X., Zhong P., Yan Z., Dopamine D₄ receptors modulate GABAergic signaling in pyramidal neurons of prefrontal cortex, J. Neurosci., 2002, 22, 9185-9193
Yan Z., Regulation of GABAergic inhibition by serotonin signaling in prefrontal cortex: molecular mechanisms and functional implications, Mol. Neurobiol., 2002, 26, 203-216

1. A method for identifying a compound or a combination of compounds having a pharmacological behavior that qualifies it as a candidate for clinical development of a drug for treatment of a psychiatric disease or disorder, said method comprising:
(i) treating neuronal cells expressing elements of the dopaminergic, gamma aminobutyric acid (GABA)-ergic and serotonergic systems with (a) said compound or combination of compounds; (b) a drug or drug combination effective against both positive and negative symptoms of psychiatric diseases or disorders; or (c) a control vehicle, for a sufficient time period;
(ii) measuring parameters selected from levels of proteins encoded by genes associated with expression or regulation of the GABA system, or phosphorylation levels of said proteins, in lysates or fractions thereof, obtained from said neuronal cells treated according to (i-a), (i-b) and (i-c), thus obtaining a test biochemical profile expressing the differences in said parameters between the neuronal cells treated according to (i-a) and the neuronal cells treated according to (i-c), and a reference biochemical profile expressing the differences in said parameters between the neuronal cells treated according to (i-b) and the neuronal cells treated according to (i-c); and
(iii) comparing said test biochemical profile with said reference biochemical profile,
wherein a significant similarity between said test biochemical profile and said reference biochemical profile indicates that said compound or combination of compounds has a likelihood of being a suitable candidate for clinical development of a drug for treatment of said psychiatric disease or disorder.

2. The method of claim 1, wherein said neuronal cells are cortical neuronal cell culture or neuronal cells obtained from a cortex, preferably a frontal cortex, more preferably a prefrontal cortex, of a mammal.

3. The method of claim 1, wherein said drug combination effective against both positive and negative symptoms of psychiatric diseases or disorders is a combination of an antipsychotic agent and an antidepressant agent functioning pharmacologically as a selective serotonin reuptake inhibitor (SSRI).

4. The method of claim 3, wherein said antipsychotic agent is selected from the group consisting of risperidone, olanzapine, ziprasidone, clozapine, haloperidol, perphenazine, trifluperazine, amisulpride, chlorprothixene, thiothixene, flupentixol and zuclopenthixol, and said antidepressant agent is fluvoxamine or fluoxetine.

5. The method of claim 4, wherein said drug or drug combination effective against both positive and negative symptoms of psychiatric diseases or disorders is clozapine or a combination of haloperidol and fluvoxamine.

6. The method of claim 1, wherein said genes associated with expression or regulation of the GABA system are selected from the group consisting of GABA_{A} Rβ3, GAD67, a protein kinase C (PKC) isoform, preferably PKCβ and PKCγ, ERK1, ERK2, Rack1, GSK-3, a protein kinase A (PKA) isoform, 5-HT receptor (5-HTR), DA receptor (DAR), metabotropic glutamate receptor (mGLUR), N-methyl-D-aspartate receptor (NMDAR), adenylate cyclase (AC), diacylglycerol (DAG), and phospholipase C (PLC).

7. The method of claim 6, wherein said genes associated with expression or regulation of the GABA system are GABA_{A} Rβ3, PKCβ2, ERK1 and ERK2, and said parameters include total GABA_{A} Rβ3 protein level, cytosolic fraction GABA_{A} Rβ3 protein level, membranal fraction GABA_{A} Rβ3 protein level, total GABA_{A} Rβ3 phosphorylation level, total PKCβ2 protein level, total ERK1 protein level, total ERK1 phosphorylation level, total ERK2 protein level and total ERK2 phosphorylation level.

8. The method of claim 7, wherein said reference biochemical profile comprises a decrease in the total GABA_{A} Rβ3 protein level; an increase in the total GABA_{A} Rβ3 phosphorylation level; a decrease in the membranal fraction GABA_{A} Rβ3 protein level; an increase in the cytosolic fraction GABA_{A} Rβ3 protein level; a decrease in the total PKCβ2 protein level; an increase in both the total ERK1 and the total ERK2 protein levels; and a decrease in both the total ERK1 and the total ERK2 phosphorylation levels, and said neuronal cells are cortical neuronal cell culture treated with said drug or drug combination for a time period of about 7 days or more, or neuronal cells obtained from a cortex, preferably a frontal cortex, more preferably a prefrontal cortex, of a mammal administered with said drug or drug combination for a time period of about 14 days or more.

9. The method of claim 1, wherein said psychiatric disease or disorder is selected from the group consisting of schizophrenia, obsessive-compulsive disorder (OCD), major depression, bipolar disorder or dementia that may be accompanied or complicated by affective disorder or aggression.

10. The method of claim 9, wherein said psychiatric disease or disorder is schizophrenia.

11. A method for identifying a compound or a combination of compounds having a pharmacological behavior that qualifies it as a candidate for clinical development of a drug for treatment of schizophrenia, said method comprising:
(i) treating neuronal cells expressing elements of the dopaminergic, GABAergic and serotonergic systems with (a) said compound or combination of compounds; or (b) a control vehicle, wherein said neuronal cells are cortical neuronal cell culture treated for a time period of about 7 days or more, or neuronal cells obtained from a cortex, preferably a frontal cortex, more preferably a prefrontal cortex, of a mammal administered with (a) or (b), for a time period of about 14 days or more;
(ii) measuring total levels of proteins encoded by the genes GABA_{A} Rβ3, PKCβ2, ERK1 and ERK2, cytosolic fraction GABA_{A} Rβ3 protein level, membranal fraction GABA_{A} Rβ3 protein level, and phosphorylation levels of GABA_{A} Rβ3, ERK1 and ERK2 in lysates, or fractions thereof, obtained from said neuronal cells treated according to (i-a) and (i-b); and
(iii) comparing the levels obtained in (ii) for the neuronal cells treated according to (i-a) and for the neuronal cells treated according to (i-b),
wherein a decrease in the total GABA_{A} Rβ3 protein level; an increase in the total GABA_{A} Rβ3 phosphorylation level; a decrease in the membranal fraction GABA_{A} Rβ3 protein level; an increase in the cytosolic fraction GABA_{A} Rβ3 protein level; a decrease in the total PKCβ2 protein level; an increase in both the total ERK1 and the total ERK2 protein levels; and a decrease in both the total ERK1 and the total ERK2 phosphorylation levels in the neuronal cells treated according to (i-a) in comparison to that of the neuronal cells treated according to (i-b) indicate that said compound or combination of compounds has a likelihood of being a suitable candidate for clinical development of a drug for treatment of schizophrenia.

12. A kit for determining whether a compound or a combination of compounds has a pharmacological behavior that qualifies it as a candidate for clinical development of a drug for treatment of a psychiatric disease or disorder, said kit comprising:
(i) a list of parameters selected from levels of proteins encoded by genes associated with expression or regulation of the GABA system, or phosphorylation levels of said proteins;
(ii) a predetermined reference biochemical profile expressing the differences in said parameters in neuronal cells expressing elements of the dopaminergic, gamma aminobutyric acid (GABA)-ergic and serotonergic systems, treated for a sufficient time period with a drug or drug combination effective against both positive and negative symptoms of psychiatric diseases or disorders as compared with a control vehicle;
(iii) a container containing said drug or drug combination;
(iv) a set of reagents required for the detection and quantification of said parameters in neuronal cells expressing elements of the dopaminergic, gamma aminobutyric acid (GABA)-ergic and serotonergic systems, said set of reagents comprising: (a) a blotting membrane; (b) a blocking agent; (c) a primary antibody against each one of said proteins or phosphorylated form of said proteins;
(d) a secondary antibody against each one of said primary antibodies, wherein said secondary antibody is linked to a detectable label; and optionally (e) a substrate for the detection of said label; and
(v) instructions for use.

13. The kit of claim 12, wherein said neuronal cells are cortical neuronal cell culture or neuronal cells obtained from a cortex, preferably a frontal cortex, more preferably a prefrontal cortex, of a mammal.

14. The kit of claim 12, wherein said drug combination effective against both positive and negative symptoms of psychiatric diseases or disorders is a combination of an antipsychotic agent and an antidepressant agent functioning pharmacologically as a selective serotonin reuptake inhibitor (SSRI).

15. The kit of claim 14, wherein said antipsychotic agent is selected from the group consisting of risperidone, olanzapine, ziprasidone, clozapine, haloperidol, perphenazine, trifluperazine, amisulpride, chlorprothixene, thiothixene, flupentixol and zuclopenthixol, and said antidepressant agent is fluvoxamine or fluoxetine.

16. The kit of claim 15, wherein said drug or drug combination effective against both positive and negative symptoms of psychiatric diseases or disorders is clozapine or a combination of haloperidol and fluvoxamine.

17. The kit of claim 12, wherein said genes associated with expression or regulation of the GABA system are selected from the group consisting of GABA_{A} Rβ3, GAD67, a protein kinase C (PKC) isoform, preferably PKCβ and PKCγ, ERK1, ERK2, Rack1, GSK-3, a protein kinase A (PKA) isoform, 5-HT receptor (5-HTR), DA receptor (DAR), metabotropic glutamate receptor (mGLUR), N-methyl-D-aspartate receptor (NMDAR), adenylate cyclase (AC), diacylglycerol (DAG), and phospholipase C (PLC).

18. The kit of claim 17, wherein said genes associated with expression or regulation of the GABA system are GABA_{A} Rβ3, PKCβ2, ERK1 and ERK2, and said parameters include total GABA_{A} Rβ3 protein level, cytosolic fraction GABA_{A} Rβ3 protein level, membranal fraction GABA_{A} Rβ3 protein level, total GABA_{A} Rβ3 phosphorylation level, total PKCβ2 protein level, total ERK1 protein level, total ERK1 phosphorylation level, total ERK2 protein level and total ERK2 phosphorylation level.

19. The kit of claim 18, wherein said predetermined reference biochemical profile comprises a decrease in the total GABA_{A} Rβ3 protein level; an increase in the total GABA_{A} Rβ3 phosphorylation level; a decrease in the membranal fraction GABA_{A} Rβ3 protein level; an increase in the cytosolic fraction GABA_{A} Rβ3 protein level; a decrease in the total PKCβ2 protein level; an increase in both the total ERK1 and the total ERK2 protein levels; and a decrease in both the total ERK1 and the total ERK2 phosphorylation levels, and said neuronal cells are cortical neuronal cell culture treated with said drug or drug combination for a time period of about 7 days or more, or neuronal cells obtained from a cortex, preferably a frontal cortex, more preferably a prefrontal cortex, of a mammal administered with said drug or drug combination for a time period of about 14 days or more.

20. The kit of claim 12, wherein said psychiatric disease or disorder is selected from the group consisting of schizophrenia, obsessive-compulsive disorder (OCD), major depression, bipolar disorder or dementia that may be accompanied or complicated by affective disorder or aggression.

21. The kit of claim 20, wherein said psychiatric disease or disorder is schizophrenia.
